(19) United States
(12) Patent Application Publication
(10) Pub. No.: US 2002/0103136 A1
(43) Pub. Date: Aug. 1, 2002

(54) Conjugates useful in the treatment of prostate cancer

(76) Inventor: Dong-Mei Feng, Blue Bell, PA (US)

Correspondence Address: MERCK AND CO INC, P O BOX 2000, RAHWAY, NJ, 070650907

(21) Appl. No.: 09/924,824

(22) Filed: Aug. 8, 2001

Related U.S. Application Data
(63) Continuation of application No. 09/262,538, filed on Mar. 4, 1999.
(60) Provisional application No. 60/076,860, filed on Mar. 5, 1998.

(51) Int. Cl. A61K 38/08; C07K 7/06

(52) U.S. Cl. 514/17; 530/330

(57) ABSTRACT

Chemical conjugates which comprise oligopeptides, having amino acid sequences that are selectively proteolytically cleaved by free prostate specific antigen (PSA) and known cytotoxic agents are disclosed. The conjugates of the invention are characterized by a hydroxylalkylamino linker between the oligopeptide and vinblastine. Such conjugates are useful in the treatment of prostatic cancer and benign prostatic hypertrophy (BPH). Also disclosed are novel cytotoxic agents that are derivatives of vinca alkaloid drugs.

### RELATED APPLICATION

The present patent application claims priority from copending provisional application Ser. No. 60/076,860, filed Mar. 5, 1998.

### BACKGROUND OF THE INVENTION

In 1996 cancer of the prostate gland was expected to be diagnosed in 317,000 men in the U.S. and 42,000 American males die from this disease (Garnick, M. B. (1994). The Dilemmas of Prostate Cancer. Scientific American, April:72-81). Thus, prostate cancer is the most frequently diagnosed malignancy (other than that of the skin) in U.S. men and the second leading cause of cancer-related deaths (behind lung cancer) in that group.

Prostate specific Antigen (PSA) is a single chain 33 kDa glycoprotein that is produced almost exclusively by the human prostate epithelium and occurs at levels of 0.5 to 2.0 mg/ml in human seminal fluid (Nadji, M., Taber, S. Z., Castro, A., et al. (1981) Cancer 48:1229; Papsidero, L., Kuriyama, M., Wang, M., et al. (1981). JNCI 66:37; Qui, S. D., Young, C. Y. F., Bihartz, D. L., et al. (1990), J. Urol. 144:1550; Wang, M. C., Valenzuela, L. A., Murphy, G. P., et al. (1979). Invest. Urol. 17:159). The single carbohydrate unit is attached at asparagine residue number 45 and accounts for 2 to 3 kDa of the total molecular mass. PSA is a protease with chymotrypsin-like specificity (Christensson, A., Laurell, C. B., Lilja, H. (1990). Eur. J. Biochem. 194:755-763). It has been shown that PSA is mainly responsible for dissolution of the gel structure formed at ejaculation by proteolysis of the major proteins in the sperm entrapping gel, Semenogelin I and Semenogelin II, and fibronectin (Lilja, H. (1985). J. Clin. Invest. 76:1899; Lilja, H., Oldbring, J., Rannevik, G., et al. (1987). J. Clin. Invest. 80:281; McGee, R. S., Herr, J. C. (1988). Biol. Reprod. 39:499). The PSA mediated proteolysis of the gel-forming proteins generates several soluble Semenogelin I and Semenogelin II fragments and soluble fibronectin fragments with liquefaction of the ejaculate and release of progressively motile spermatoza (Lilja, H., Laurell, C. B. (1984). Scand. J. Clin. Lab. Invest. 44:447; McGee, R. S., Herr, J. C. (1987). Biol. Reprod. 37:431). Furthermore, PSA may proteolytically degrade IGFBP-3 (insulin-like growth factor binding protein 3) allowing IGF to stimulate specifically the growth of PSA secreting cells (Cohen et al., (1992) J. Clin. Endo. & Meta. 75:1046-1053).

PSA complexed to alpha 1-antichymotrypsin is the predominant molecular form of serum PSA and may account for up to 95% of the detected serum PSA (Christensson, A., Bjork, T., Nilsson, O., et al. (1993). J. Urol. 150:100-105; Lilja, H., Christensson, A., Dahlén, U. (1991). Clin. Chem. 37:1618-1625; Stenman, U. H., Leinoven, J., Alfthan, H., et al. (1991). Cancer Res. 51:222-226). The prostatic tissue (normal, benign hyperplastic, or malignant tissue) is implicated to predominantly release the mature, enzymatically active form of PSA, as this form is required for complex formation with alpha 1-antichymotrypsin (Mast, A. E., Enghild, J. J., Pizzo, S. V., et al. (1991). Biochemistry 30:1723-1730; Perlmutter, D. H., Glover, G. I., Rivetna, M., et al. (1990). Proc. Natl. Acad. Sci. USA 87:3753-3757). Therefore, in the microenvironment of prostatic PSA secreting cells the PSA is believed to be processed and secreted in its mature enzymatically active form not complexed to any inhibitory molecule. PSA also forms stable complexes with alpha 2-macroglobulin, but as this results in encapsulation of PSA and complete loss of the PSA epitopes, the in vivo significance of this complex formation is unclear. A free, noncomplexed form of PSA constitutes a minor fraction of the serum PSA (Christensson, A., Bjork, T., Nilsson, O., et al. (1993). J. Urol. 150:100-105; Lilja, H., Christensson, A., Dahlén, U. (1991). Clin. Chem. 37:1618-1625). The size of this form of serum PSA is similar to that of PSA in seminal fluid (Lilja, H., Christensson, A., Dahlén, U. (1991). Clin. Chem. 37:1618-1625) but it is yet unknown as to whether the free form of serum PSA may be a zymogen; an internally cleaved, inactive form of mature PSA; or PSA manifesting enzyme activity. However, it seems unlikely that the free form of serum PSA manifests enzyme activity, since there is considerable (100 to 1000 fold) molar excess of both unreacted alpha 1-antichymotrypsin and alpha 2-macroglobulin in serum as compared with the detected serum levels of the free 33 kDa form of PSA (Christensson, A., Bjork, T., Nilsson, O., et al. (1993). J. Urol. 150:100-105; Lilja, H., Christensson, A., Dahlén, U. (1991). Clin. Chem. 37:1618-1625).

Serum measurements of PSA are useful for monitoring the treatment of adenocarcinoma of the prostate (Duffy, M. S. (1989). Ann. Clin. Biochem. 26:379-387; Brawer, M. K. and Lange, P. H. (1989). Urol. Suppl. 5:11-16; Hara, M. and Kimura, H. (1989). J. Lab. Clin. Med. 113:541-548), although above normal serum concentrations of PSA have also been reported in benign prostatic hyperplasia and subsequent to surgical trauma of the prostate (Lilja, H., Christensson, A., Dahlén, U. (1991). Clin. Chem. 37:1618-1625). Prostate metastases are also known to secrete immunologically reactive PSA since serum PSA is detectable at high levels in prostatectomized patients showing widespread metatstatic prostate cancer (Ford, T. F., Butcher, D. N., Masters, R. W., et al. (1985). Brit. J. Urology 57:50-55). Therefore, a cytotoxic compound that could be activated by the proteolytic activity of PSA should be prostate cell specific as well as specific for PSA secreting prostate metastases.

U.S. Pat. No. 4,203,898 describes derivative of the vinca alkaloid cytotoxic agents wherein the C-3 methyl ester of the vinca drug has been modified.

It is the object of this invention to provide a novel anti-cancer composition useful for the treatment of prostate cancer which comprises oligopeptides, that are selectively proteolytically cleaved by free prostate specific antigen (PSA) and that are linked, via a hydroxylalkylamino linker, to a cytotoxic agent.

Another object of this invention is to provide a method of treating prostate cancer which comprises administration of the novel anti-cancer composition.

A further object of the invention is to provide novel cytotoxic derivatives of vinca alkaloid cytotoxic agents.

### SUMMARY OF THE INVENTION

Chemical conjugates which comprise oligopeptides, having amino acid sequences that are selectively proteolytically cleaved by free prostate specific antigen (PSA), and a cytotoxic agent are disclosed. The conjugates of the invention are characterized by a hydroxyalkylamine linker between the oligopeptide and a vinca alkaloid drug. Such conjugates are useful in the treatment of prostatic cancer and benign prostatic hyperplasia (BPH). Also disclosed are novel cytotoxic derivatives of vinca alkaloid drugs wherein the C-23 ester of the vinca alkaloid is replaced with an unsubstituted or suitably substituted hydroxyalkylamide.

### DETAILED DESCRIPTION OF THE INVENTION

The instant invention relates to novel anti-cancer compositions useful for the treatment of prostate cancer. Such compositions comprise the oligopeptides covalently bonded through a chemical linker to cytotoxic agent, preferably a vinca drug. The oligopeptides are chosen from oligomers that are selectively recognized by the free prostate specific antigen (PSA) and are capable of being proteolytically cleaved by the enzymatic activity of the free prostate specific antigen. Such a combination of an oligopeptide and cytotoxic agent may be termed a conjugate.

The conjugates of the instant invention are characterized by a linker between the C-terminus of the oligopeptide and the vinca drug. In particular, the linker is a hydroxyalkylamine moiety, which is optionally substituted, and most preferably, the linker comprises a sterically hindered hydroxyalkylamine moiety. Also preferably, the attachment of the oligopeptide to the linker is through an ester bond with the hydroxyl moiety of the linker.

Ideally, the cytotoxic activity of the vinca drug is greatly reduced or absent when the oligopeptide containing the PSA proteolytic cleavage site is bonded through the chemical linker to the cytotoxic agent and is intact. Also ideally, the cytotoxic activity of the cytotoxic agent increases significantly or returns to the activity of the unmodified cytotoxic agent upon proteolytic cleavage of the attached oligopeptide at the cleavage site.

Preferably, the vinca drug with the chemical linker intact exhibits cytotoxic activity that is at least 75% of the cytotoxicity of the unmodified vinca drug against the target cancer cells. Such a derivative of the vinca drug wherein the chemical linker is still covalently bound to the vinca drug may itself be considered a cytotoxic agent.

Furthermore, it is preferred that the oligopeptide is selected from oligopeptides that are not cleaved or are cleaved at a much slower rate in the presence of non-PSA proteolytic enzymes, such as those enzymes endogenous to human serum, when compared to the cleavage of the oligopeptides in the presence of free enzymatically active PSA.

For the reasons above, it is desirable for the oligopeptide to comprise a short peptide sequence, preferably less than ten amino acids. Most preferably the oligopeptide comprises seven or six amino acids. Because the conjugate preferably comprises a short amino acid sequence, the solubility of the conjugate may be influenced to a greater extent by the generally hydrophobic character of the cytotoxic agent component. Therefore, amino acids with hydrophilic substituents may be incorporated in the oligopeptide sequence or N-terminus blocking groups may be selected to offset or diminish such a hydrophobic contribution by the cytotoxic agent. Combinations of amino acids with hydrophilic substituents and N-terminus blocking groups that enhance solubility may also be employed in a single conjugate.

While it is not necessary for practicing this aspect of the invention, an embodiment of this invention is a conjugate wherein the oligopeptide and the chemical linker are detached from the cytotoxic agent by the proteolytic activity of the free PSA and any other native proteolytic enzymes present in the tissue proximity, thereby presenting the cytotoxic agent, or a cytotoxic agent that retains part of the oligopeptide/linker unit but remains cytotoxic, into the physiological environment at the place of proteolytic cleavage. Pharmaceutically acceptable salts of the conjugates are also included.

It is understood that the oligopeptide, that is conjugated to the cytotoxic agent through a chemical linker, does not need to be the oligopeptide that has the greatest recognition by free PSA and is most readily proteolytically cleaved by free PSA. Thus, the oligopeptide that is selected for incorporation in such an anti-cancer composition will be chosen both for its selective, proteolytic cleavage by free PSA and for the cytotoxic activity of the cytotoxic agent-proteolytic residue conjugate (or, in what is felt to be an ideal situation, the unmodified cytotoxic agent) which results from such a cleavage. The term “selective” as used in connection with the proteolytic PSA cleavage means a greater rate of cleavage of an oligopeptide component of the instant invention by free PSA relative to cleavage of an oligopeptide which comprises a random sequence of amino acids. Therefore, the oligopeptide component of the instant invention is a prefered substrate of free PSA. The term “selective” also indicates that the oligopeptide is proteolytically cleaved by free PSA between two specific amino acids in the oligopeptide.

The oligopeptide components of the instant invention are selectively recognized by the free prostate specific antigen (PSA) and are capable of being proteolytically cleaved by the enzymatic activity of the free prostate specific antigen. Such oligopeptides comprise an oligomer selected from:

a) AsnLysIIeSerTyrGln/Ser (SEQ.ID.NO.: 1),

b) LysIleSerTyrGln/Ser (SEQ.ID.NO.: 2),

c) AsnLysIleSerTyrTyr/Ser (SEQ.ID.NO.: 3),

d) AsnLysAlaSerTyrGln/Ser (SEQ.ID.NO.: 4),

e) SerTyrGln/SerSer (SEQ.ID.NO.: 5);

f) LysTyrGln/SerSer (SEQ.ID.NO.: 6);

g) hArgTyrGln/SerSer (SEQ.ID.NO.: 7);

h) hArgChaGln/SerSer (SEQ.ID.NO.: 8);

i) TyrGln/SerSer (SEQ.ID.NO.: 9);

j) TyrGln/SerLeu (SEQ.ID.NO.: 10);

k) TyrGln/SerNle (SEQ.ID.NO.: 11);

l) ChgGln/SerLeu (SEQ.ID.NO.: 12);

m) ChgGln/SerNle (SEQ.ID.NO.: 13);

n) SerTyrGln/Ser (SEQ.ID.NO.: 14);

o) SerChgGln/Ser (SEQ.ID.NO.: 15);

p) SerTyrGln/SerVal (SEQ.ID.NO.: 16);

q) SerChgGln/SerVal (SEQ.ID.NO.: 17);

r) SerTyrGln/SerLeu (SEQ.ID.NO.: 18);

s) SerChgGln/SerLeu (SEQ.ID.NO.: 19);

t) HaaXaaSerTyrGln/Ser (SEQ.ID.NO.: 20);

u) HaaXaaLysTyrGln/Ser (SEQ.ID.NO.: 21);

v) HaaXaahArgTyrGln/Ser (SEQ.ID.NO.: 22);

w) HaaXaahArgChaGln/Ser (SEQ.ID.NO.: 23);

x) HaaTyrGln/Ser (SEQ.ID.NO.: 24);

y) HaaXaaSerChgGln/Ser (SEQ.ID.NO.: 25);

z) HaaChgGln/Ser (SEQ.ID.NO.: 26);

aa) SerChgGln/SerSer (SEQ.ID.NO.: 106);

bb) SerChgGln/SerPro (SEQ.ID.NO.: 107);

cc) SerChgGln/SerAbu (SEQ.ID.NO.: 108);

wherein Haa is a cyclic amino acid substituted with a hydrophilic moiety, hArg is homoarginine, Xaa is any amino acid, Cha is cyclohexylalanine, Abu is 2-aminobutyric acid and Chg is cyclohexylglycine.

In an embodiment of the instant invention, the oligopeptide comprises an oligomer that is selected from:

a) SerSerTyrGln/SerVal (SEQ.ID.NO.: 27);

b) SerSerChgGln/SerVal (SEQ.ID.NO.: 28);

c) SerSerTyrGln/SerLeu (SEQ.ID.NO.: 29);

e) SerSerChgGln/SerLeu (SEQ.ID.NO.: 30);

f) SerSerTyrGln/SerSer (SEQ.ID.NO.: 31);

g) SerSerChgGln/SerSer (SEQ.ID.NO.: 32);

h) SerSerTyrGln/SerPro (SEQ.ID.NO.: 33);

i) SerSerChgGln/SerPro (SEQ.ID. NO.: 34);

j) 4-HypSerSerTyrGln/Ser (SEQ.ID.NO.: 35);

k) 4-HypSerSerChgGln/Ser (SEQ.ID.NO.: 36);

l) AlaSerTyrGln/SerVal (SEQ.ID.NO.: 37);

m) AlaSerChgGln/SerVal (SEQ.ID.NO.: 38);

n) AlaSerTyrGln/SerLeu (SEQ.ID.NO.: 39);

o) AlaSerChgGln/SerLeu (SEQ.ID.NO.: 40);

p) 4-HypAlaSerTyrGln/Ser (SEQ.ID.NO.: 41);

q) 4-HypAlaSerChgGln/Ser (SEQ.ID.NO.: 42);

wherein 4-Hyp is 4-hydroxyproline, Xaa is any amino acid, hArg is homoarginine, Cha is cyclohexylalanine and Chg is cyclohexylglycine.

In a more preferred embodiment of the instant invention, the oligopeptide comprises an oligomer selected from:

SerSerChgGln/SerLeu (SEQ.ID.NO.: 43);

SerSerChgGln/SerVal (SEQ.ID.NO.: 44);

SerSerChgGln/SerPro (SEQ.ID.NO.: 45);

SerSerChgGln/SerSer (SEQ.ID.NO.: 46);

SerSerSerChgGln/SerLeu (SEQ.ID.NO.: 47);

SerSerSerChgGln/SerVal (SEQ.ID.NO.: 48);

SerSerSerChgGln/SerPro (SEQ.ID.NO.: 49);

SerSerSerChgGln/SerSer (SEQ.ID.NO.: 50);

SerAlaSerChgGln/SerLeu (SEQ.ID.NO.: 51);

SerAlaSerChgGln/SerVal (SEQ.ID.NO.: 52);

(N-methyl-Ser)SerSerChgGln/SerLeu (SEQ.ID.NO.: 53);

(N-methyl-Ser)SerSerChgGln/SerVal (SEQ.ID.NO.: 54);

4-HypSerSerTyrGln/SerVal (SEQ.ID.NO.: 55);

4-HypSerSerTyrGln/SerLeu (SEQ.ID.NO.: 56);

4-HypSerSerChgGln/SerVal (SEQ.ID.NO.: 57);

4-HypSerSerChgGln/SerLeu (SEQ.ID.NO.: 58);

4-HypSerSerChgGln/SerSer (SEQ.ID.NO.: 59);

4-HypSerSerChgGln/SerSer (SEQ.ID.NO.: 60);

4-HypSerSerChgGln/SerPro (SEQ.ID.NO.: 61);

4-HypSerSerChgGln/SerPro (SEQ.ID.NO.: 62);

4-HypAlaSerChgGln/SerVal (SEQ.ID.NO.: 63);

4-HypAlaSerChgGln/SerLeu (SEQ.ID.NO.: 64);

(3,4-DiHyp)SerSerTyrGln/SerVal (SEQ.ID.NO.: 65); and

(3,4-DiHyp)SerSerTyrGln/SerLeu (SEQ.ID.NO.: 66);

wherein 4-Hyp is 4-hydroxyproline, 3,4-DiHyp is 3,4-dihydroxyproline and Chg is cyclohexylglycine.

The phrase “oligomers that comprise an amino acid sequence” as used hereinabove, and elsewhere in the Detailed Description of the Invention, describes oligomers of from about 3 to about 100 amino acids residues which include in their amino acid sequence the specific amino acid sequence decribed and which are therefore proteolytically cleaved within the amino acid sequence described by free PSA. Preferably, the oligomer is from 5 to 10 amino acid residues. Thus, for example, the following oligomer: hArgSerAlaChgGln/SerLeu (SEQ.ID.NO.: 67); comprises the amino acid sequence: ChgGln/SerLeu (SEQ.ID.NO.: 12); and would therefore come within the instant invention. And the oligomer: hArgSer4-HypChgGln/SerLeu (SEQ.ID.NO.: 68); comprises the amino acid sequence: 4-HypChgGln/SerLeu (SEQ.ID.NO.: 69); and would therefore come within the instant invention. It is understood that such oligomers do not include semenogelin I and semenogelin II.

A person of ordinary skill in the peptide chemistry art would readily appreciate that certain amino acids in a biologically active oligopeptide may be replaced by other homologous, isosteric and/or isoelectronic amino acids wherein the biological activity of the original oligopeptide has been conserved in the modified oligopeptide. Certain unnatural and modified natural amino acids may also be utilized to replace the corresponding natural amino acid in the oligopeptides of the instant invention. Thus, for example, tyrosine may be replaced by 3-iodotyrosine, 2-methyltyrosine, 3-fluorotyrosine, 3-methyltyrosine and the like. Further for example, lysine may be replaced with N′-(2-imidazolyl)lysine and the like. The following list of amino acid replacements is meant to be illustrative and is not limiting:

| Original Amino Acid | Replacement Amino Acid(s) |
|---|---|
| Ala | Gly |
| Arg | Lys, Ornithine |
| Asn | Gln |
| Asp | Glu |
| Glu | Asp |
| Gln | Asn |
| Gly | Ala |
| Ile | Val, Leu, Met, Nle |
| Leu | Ile, Val, Met, Nle |
| Lys | Arg, Ornithine |
| Met | Leu, Ile, Nle, Val |
| Ornithine | Lys, Arg |
| Phe | Tyr, Trp |
| Ser | Thr |
| Thr | Ser |
| Trp | Phe, Tyr |
| Tyr | Phe, Trp |
| Val | Leu, Ile, Met, Nle |

Thus, for example, the following oligopeptides may be synthesized by techniques well known to persons of ordinary skill in the art and would be expected to be proteolytically cleaved by free PSA:

AsnArgIleSerTyrGln/Ser (SEQ.ID.NO.: 70)

AsnLysValSerTyrGln/Ser (SEQ.ID.NO.: 71)

AsnLysMetSerTyrGln/SerSer (SEQ.ID.NO.: 72)

AsnLysLeuSerTyrGln/SerSer (SEQ.ID.NO.: 73)

AsnLysIleSerTyrGln/Ser (SEQ.ID.NO.: 74)

GlnLysIleSerTyrGln/SerSer (SEQ.ID.NO.: 75).

Asn4-HypIleSerTyrGln/Ser (SEQ.ID.NO.: 76)

Asn4-HypValSerTyrGln/Ser (SEQ.ID.NO.: 77)

4-HypAlaSerTyrGln/SerSer (SEQ.ID.NO.: 78)

(3,4-dihydroxyproline)AlaSerTyrGln/SerSer (SEQ.ID.NO.: 79)

3-hydroxyprolineSerChgGln/Ser (SEQ.ID.NO.: 80)

4-HypAlaSerChgGln/SerSer (SEQ.ID.NO.: 81).

The inclusion of the symbol “I” within an amino acid sequence indicates the point within that sequence where the oligopeptide is proteolytically cleaved by free PSA.

The compounds of the present invention may have asymmetric centers and occur as racemates, racemic mixtures, and as individual diastereomers, with all possible isomers, including optical isomers, being included in the present invention. Unless otherwise specified, named amino acids are understood to have the natural “L” stereoconfiguration

In the present invention, the amino acids which are disclosed are identified both by conventional 3 letter and single letter abbreviations as indicated below:

| | | |
|---|---|---|
| Alanine | Ala | A |
| Arginine | Arg | R |
| Asparagine | Asn | N |
| Aspartic acid | Asp | D |
| Asparagine or | Asx | B |
| Aspartic acid | | |
| Cysteine | Cys | C |
| Glutamine | Gln | Q |
| Glutamic acid | Glu | E |
| Glutamine or | Glx | Z |
| Glutamic acid | | |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The following abbreviations are utilized in the specification and figures to denote the indicated amino acids and moieties:

| | |
|---|---|
| hR or hArg: | homoarginine |
| hY or hTyr: | homotyrosine |
| Cha: | cyclohexylalanine |
| Amf: | 4-aminomethylphenylalanine |
| DAP: | 1,3-diaminopropyl |
| DPL: | 2-(4,6-dimethylpyrimidinyl)lysine |
| (imidazolyl)K: | N′-(2-imidazolyl)lysine |
| Me₂PO₃-Y: | O-dimethylphosphotyrosine |
| O—Me-Y: | O-methyltyrosine |
| TIC: | 1,2,3,4-tetrahydro-3-isoquinoline carboxylic acid |
| DAP: | 1,3-diaminopropane |
| TFA: | trifluoroacetic acid |
| AA: | acetic acid |
| 3 PAL: | 3-pyridylalanine |
| 4-Hyp: | 4-hydroxyproline |
| dAc-Vin: | 4-des-acetylvinbiastine |
| Trt: | trityl |

It is well known in the art, and understood in the instant invention, that peptidyl therapeutic agents such as the instant oligopeptide-cytotoxic agent conjugates preferably have the terminal amino moiety of any oligopeptide substituent protected with a suitable protecting group, such as acetyl, benzoyl, pivaloyl and the like. Such protection of the terminal amino group reduces or eliminates the enzymatic degradation of such peptidyl therapeutic agents by the action of exogenous amino peptidases which are present in the blood plasma of warm blooded animals. Such protecting groups also include hydrophilic blocking groups, which are chosen based upon the presence of hydrophilic functionality. Blocking groups that increase the hydrophilicity of the conjugates and therefore increase the aqueous solubility of the conjugates include but are not limited to hydroylated alkanoyl, polyhydroxylated alkanoyl, polyethylene glycol, glycosylates, sugars and crown ethers. N-Terminus unnatural amino acid moieties may also ameleorate such enzymatic degradation by exogenous amino peptidases.

Preferably the N-terminus protecting group is selected from

a) acetyl;

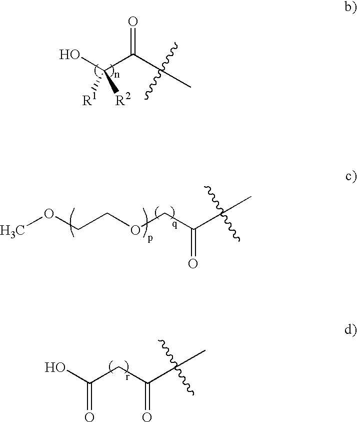

wherein:

R¹ and R² are independently selected from:

a) hydrogen,

b) unsubstituted or substituted aryl, unsubstituted or substituted heterocycle, C₃-C₁₀cycloalkyl, C₂-C₆alkenyl, C₂-C₆alkynyl, halogen, C₁-C₆perfluoroalkyl, R⁶O—, R⁶C(O)NR⁶—, (R⁶)₂NC(O)—, R⁶₂N—C(NR⁶)—, R⁷S(O)₂NH, CN, NO₂, R⁶C(O)—, N₃, —N(R⁶)₂, or R⁷OC(O)NR⁶—,

c) unsubstituted C₁-C₆alkyl,

d) substituted C₁-C₆alkyl wherein the substituent on the substituted C₁-C₆alkyl is selected from unsubstituted or substituted aryl, unsubstituted or substituted heterocyclic, C₃-C₁₀cycloalkyl, C₂-C₆alkenyl, C₂-C₆alkynyl, R⁶O—, R⁷S(O)₂NH, R⁶C(O)NR⁶—, (R⁶)₂NC(O)—, R⁶₂N—C(NR⁶)—, CN, R⁶C(O)—, N₃, —N(R⁶)₂, and R⁷OC(O)—NR⁶—; or

R¹ and R² are combined to form —(CH₂)ₛ— wherein one of the carbon atoms is optionally replaced by a moiety selected from: O, S(O)ₘ, —NC(O)—, NH and —N(COR⁷)—;

R⁶ is selected from: hydrogen, aryl, substituted aryl, heterocycle, substituted heterocycle, C₁-C₆alkyl and C₃-C₁₀cycloalkyl;

R⁷ is selected from: aryl, substituted aryl, heterocycle, substituted heterocycle, C₁-C₆alkyl and C₃-C₁₀cycloalkyl;

m is 0, 1 or 2;

n is 1, 2, 3 or 4;

p is zero or an integer between 1 and 100; and

q is 0 or 1, provided that if p is zero, q is 1; and

r is 1, 2 or 3;

s is 3, 4 or 5.

The cytotoxic agent that is utilized in the conjugates of the instant invention may be selected from alkylating agents, antiproliferative agents, tubulin binding agents and the like. Preferred classes of cytotoxic agents which may be linked to cleavable oligomers via the hydroxyalkylamine linker include, for example, the methotrexates, the vinca drugs (also known as vinca alkaloid cytotoxic agents), the mitomycins and the bleomycins. Particularly useful members of those classes include, for example, aminopterin, methotrexate, methopterin, dichloro-methotrexate, mitomycin C, porfiromycin, melphalan, vinblastine, vincristine, leurosidine, vindesine, leurosine and the like. Other useful cytotoxic agents include cisplatin and cyclophosphamide. One skilled in the art may make chemical modifications to the desired cytotoxic agent in order to make reactions of that compound more convenient for purposes of preparing conjugates of the invention.

The preferred cytotoxic agents include, in general, the vinca alkaloid cytotoxic agents. Particularly useful members of this class include, for example, vinblastine, desacetylvinblastine, vincristine, leurosidine, vindesine, vinorelbine, navelbine, leurosine and the like. One skilled in the art may make chemical modifications to the desired cytotoxic agent in order to make reactions of that compound more convenient for purposes of preparing conjugates of the invention.

The preferred group of cytotoxic agents for the present invention include drugs of the following formulae:

THE VINCA ALKALOID GROUP OF DRUGS OF FORMULA (1):

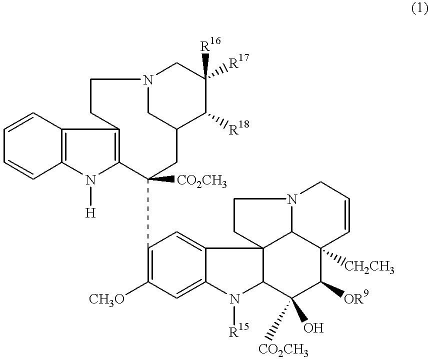

in which

R¹⁵ is H, CH₃ or CHO;

when R¹⁷ and R¹⁸ are taken singly, R¹⁸ is H, and one of R¹⁶ and R¹⁷ is ethyl and the other is H or OH;

when R¹⁷ and R¹⁸ are taken together with the carbons to which they are attached, they form an oxirane ring in which case R¹⁶ is ethyl;

R⁹ is hydrogen, (C₁-C₃alkyl)—CO, or chlorosubstituted (C₁-C₃alkyl)—CO.

The oligopeptide-cytotoxic agent conjugate of the instant invention wherein the cytotoxic agent is the preferred cytotoxic agent vinblastine may be described by the general formula I below:

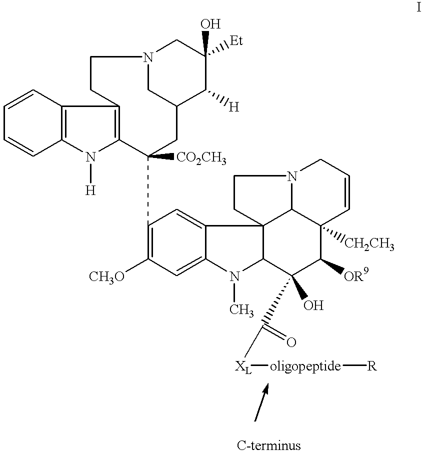

wherein:

oligopeptide is an oligopeptide which is specifically recognized by the free prostate specific antigen (PSA) and is capable of being proteolytically cleaved by the enzymatic activity of the free prostate specific antigen,

X_{L} is selected from —NH—(CR³₂)ᵤ (CR⁴₂)ᵥ—O— and

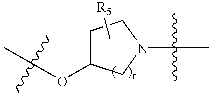

R is selected from

a) hydrogen,

b) —(C═O)R^{1a},

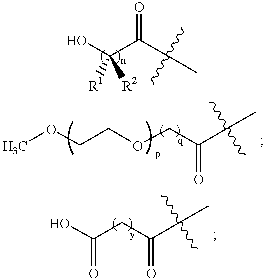

f) ethoxysquarate; and

g) cotininyl;

R¹ and R² are independently selected from:

a) hydrogen,

b) unsubstituted or substituted aryl, unsubstituted or substituted heterocycle, C₃-C₁₀cycloalkyl, C₂-C₆alkenyl, C₂-C₆alkynyl, halogen, C₁-C₆perfluoroalkyl, R⁶O—, R⁶C(O)NR⁶—, (R⁶)₂NC(O)—, R⁶₂N—C(NR⁶)—, R⁷S(O)₂NH, CN, NO₂, R⁶C(O)—, N₃, —N(R⁶)₂, or R⁷OC(O)NR⁶—,

c) unsubstituted C₁-C₆alkyl,

d) substituted C₁-C₆alkyl wherein the substituent on the substituted C₁-C₆alkyl is selected from unsubstituted or substituted aryl, unsubstituted or substituted heterocyclic, C₃-C₁₀cycloalkyl, C₂-C₆alkenyl, C₂-C₆alkynyl, R⁶O—, R⁷S(O)2NH, R⁶C(O)NR⁶—, (R⁶)₂NC(O)—, R⁶₂N—C(NR⁶)—, CN, R⁶C(O)—, N₃, —N(R⁶)₂, and R⁷OC(O)—NR⁶—; or R¹ and R² are combined to form —(CH₂)ₛ— wherein one of the carbon atoms is optionally replaced by a moiety selected from: O, S(O)ₘ, —NC(O)—, NH and —N(COR⁷)—;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl,

R³ and R⁴ are independently selected from: hydrogen, C₁-C₆-alkyl, hydroxylated C₃-C₈-cycloalkyl, polyhydroxylated C₃-C₈-cycloalkyl, hydroxylated aryl, polyhydroxylated aryl and aryl, or

one R³ and one R⁴ are combined to form a —(CH₂)_{w}—, which is unsubstituted or substituted with one or two substituents selected from OH and C₁-C₆alkyl; or

an R³ is combined with another R³ on the same carbon to form a —(CH₂)ₓ—; or

an R⁴ is combined with another R⁴ on the same carbon to form a —(CH₂)ₓ—;

R⁵ is selected from OH and C₁-C₆alkyl;

R⁶ is selected from: hydrogen, aryl, substituted aryl, heterocycle, substituted heterocycle, C₁-C₆alkyl and C₃-C₁₀cycloalkyl;

R⁷ is selected from: aryl, substituted aryl, heterocycle, substituted heterocycle, C₁-C₆alkyl and C₃-C₁₀cycloalkyl;

R⁹ is hydrogen, (C₁-C₃alkyl)—CO, or chlorosubstituted (C₁-C₃alkyl)—CO;

n is 1, 2, 3 or 4;

p is zero or an integer between 1 and 100;

q is 0 or 1, provided that if p is zero, q is 1;

r is 1, 2 or 3;

s is 4, 5 or 6;

t is 3 or 4;

u and v are independently selected from: 0, 1, 2 or 3;

w is 2, 3 or 4;

x is 3, 4 or 5;

y is 1, 2 or 3;

or the pharmaceutically acceptable salt thereof.

Preferably, u is 1 and v is 1.

Preferably, at least one R³ is selected from phenyl, cyclohexyl and cyclopentyl.

Preferably, at least one R⁴ is selected from phenyl, cyclohexyl, cyclopentyl and C₁-C₆alkyl.

Preferably, R¹ and R² are independently selected from: hydrogen, OH, C₁-C₆alkyl, C₁-C₆alkoxy, C₁-C₆aralkyl and aryl.

Preferably, attachment of the group X_{L} to the C-23 carbonyl of the vinca alkaloid cytotoxic agent is through the nitrogen of the X_{L} group.

Preferably, X_{L} is selected from the following group:

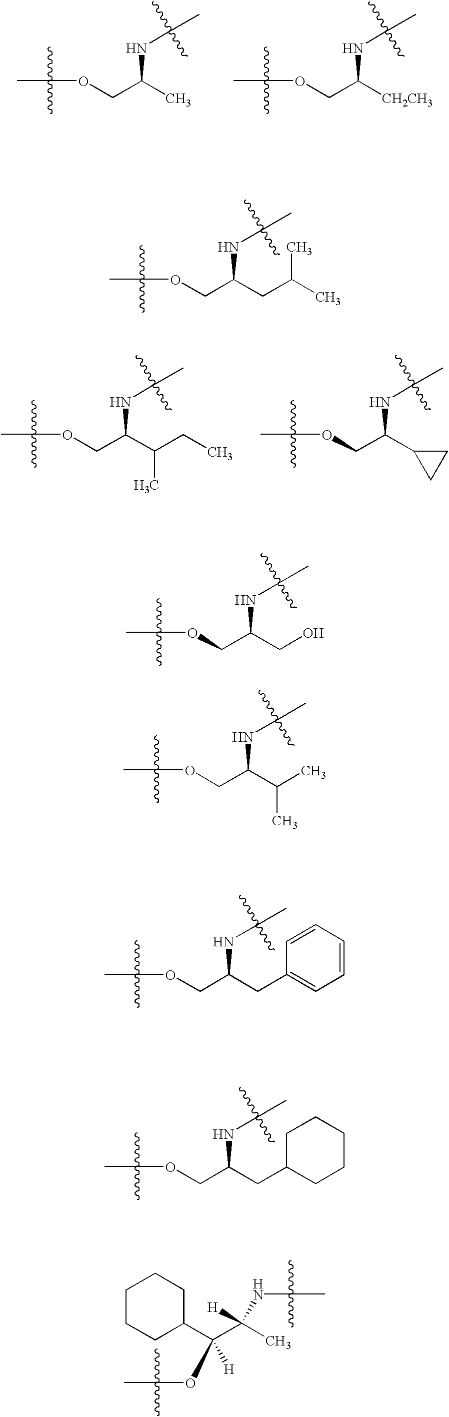

or the optical isomer thereof

More preferably, X_{L} is selected from the following group:

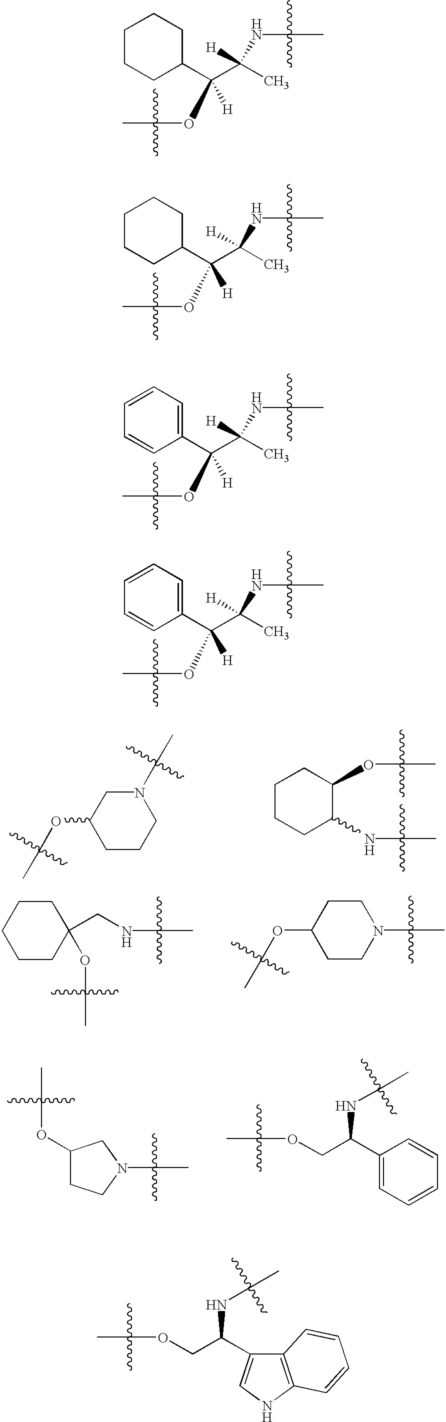

or the optical isomer thereof.

Certain of the oligopeptides of the instant conjugates comprise a cyclic amino acid substituted with a hydrophilic moiety, previously represented by the term “Haa”, which may also be represented by the formula:

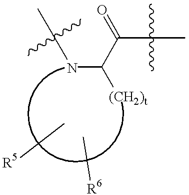

wherein:

R⁵ is selected from HO— and C₁-C₆alkoxy;

R⁶ is selected from hydrogen, halogen, C₁-C₆alkyl, HO— and C₁-C₆alkoxy; and

t is 3 or 4.

The structure

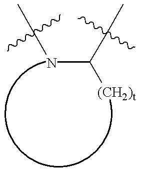

represents a cyclic amine moiety having 5 or 6 members in the ring, such a cyclic amine which may be optionally fused to a phenyl or cyclohexyl ring. Examples of such a cyclic amine moiety include, but are not limited to, the following specific structures:

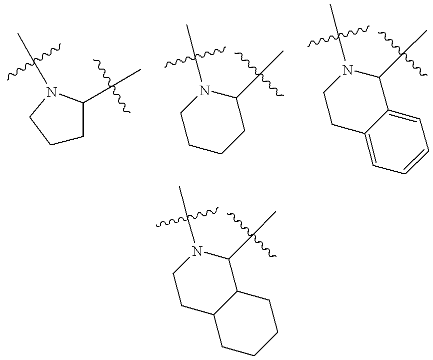

When one R³ and one R⁴ are combined to form a —(CH₂)_{w}—, a cycloalkyl moiety having 5-7 members in the ring. Examples of such cycloalkyl moieties include, but are not limited to, the following specific structures:

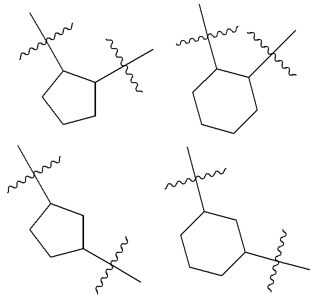

The conjugates of the present invention may have asymmetric centers and occur as racemates, racemic mixtures, and as individual diastereomers, with all possible isomers, including optical isomers, being included in the present invention. When any variable (e.g. aryl, heterocycle, R³ etc.) occurs more than one time in any constituent, its definition on each occurence is independent of every other occurence. For example, HO(CR³R³)₂— represents HOCH₂CH₂—, HOCH₂CH(OH)—, HOCH(CH₃)CH(OH)—, etc. Also, combinations of substituents and/or variables are permissible only if such combinations result in stable compounds.

As used herein, “alkyl” and the alkyl portion of aralkyl and similar terms, is intended to include both branched and straight-chain saturated aliphatic hydrocarbon groups having the specified number of carbon atoms; “alkoxy” represents an alkyl group of indicated number of carbon atoms attached through an oxygen bridge.

As used herein, “chlorosubstituted-alkyl” is intended to include both branched and straight-chain saturated aliphatic hydrocarbon groups having the specified number of carbon atoms and being substituted with a chlorine atom. Examples include, but are not limited to chloromethyl, 1-chloroethyl, 2-chloroethyl, 1-chloropropyl, 2-chloropropyl and the like.

As used herein, “cycloalkyl” is intended to include non-aromatic cyclic hydrocarbon groups having the specified number of carbon atoms. Examples of cycloalkyl groups include cyclopropyl, cyclobutyl, cyclopentyl, cyclohexyl and the like.

“Alkenyl” groups include those groups having the specified number of carbon atoms and having one or several double bonds. Examples of alkenyl groups include vinyl, allyl, isopropenyl, pentenyl, hexenyl, heptenyl, cyclopropenyl, cyclobutenyl, cyclopentenyl, cyclohexenyl, 1-propenyl, 2-butenyl, 2-methyl-2-butenyl, isoprenyl, farnesyl, geranyl, geranylgeranyl and the like.

“Alkynyl” groups include those groups having the specified number of carbon atoms and having one triple bonds. Examples of alkynyl groups include acetylene, 2-butynyl, 2-pentynyl, 3-pentynyl and the like.

“Halogen” or “halo” as used herein means fluoro, chloro, bromo and iodo.

As used herein, “aryl,” and the aryl portion of aralkyl and aroyl, is intended to mean any stable monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl.

The term heterocycle or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl.

As used herein in the terms “substituted C₁₋₈ alkyl”, “substituted aryl” and “substituted heterocycle” include moieties containing from 1 to 3 substituents in addition to the point of attachment to the rest of the compound. Such additional substituents are selected from F, Cl, Br, CF₃, NH₂, N(C₁-C₆alkyl)₂, NO₂, CN, (C₁-C₆alkyl)O—, —OH, (C₁-C₆alkyl)S(O)ₘ—, (C₁-C₆alkyl)C(O)NH—, H₂N—C(NH)—, (C₁-C₆alkyl)C(O)—, (C₁-C₆alkyl)OC(O)—, N₃, (C₁-C₆alkyl)OC(O)NH— and C₁-C₂₀alkyl.

When R¹ and R², two R³s on the same carbon, or two R⁴s on the same carbon are combined to form —(CH₂)ₛ— or —(CH₂)_{w}—, the cyclic moieties so defined include, but are not limited to:

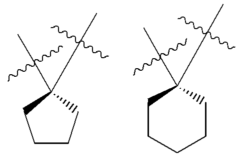

When R¹ and R² are combined to form —(CH₂)ₛ—, the heteroatom-containing cyclic moieties so defined include, but are not limited to:

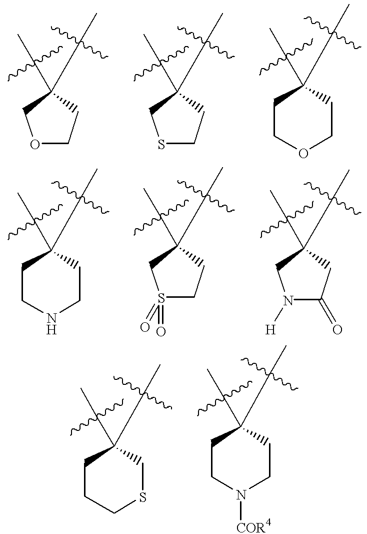

As used herein, the term “hydroxylated” represents substitution on a substitutable carbon of the ring system being so described by a hydroxyl moiety. As used herein, the term “polyhydroxylated” represents substitution on two or more substitutable carbon of the ring system being so described by two, three or four hydroxyl moieties.

As used herein, the term “cotininyl” represents the following structure:

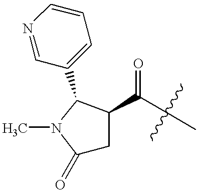

or the diastereomer thereof.

As used herein, the term “4-ethoxysquarate” represents the following structure:

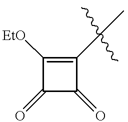

The following compounds are specific examples of the oligopeptide-desacetylvinblastine conjugate of the instant invention:

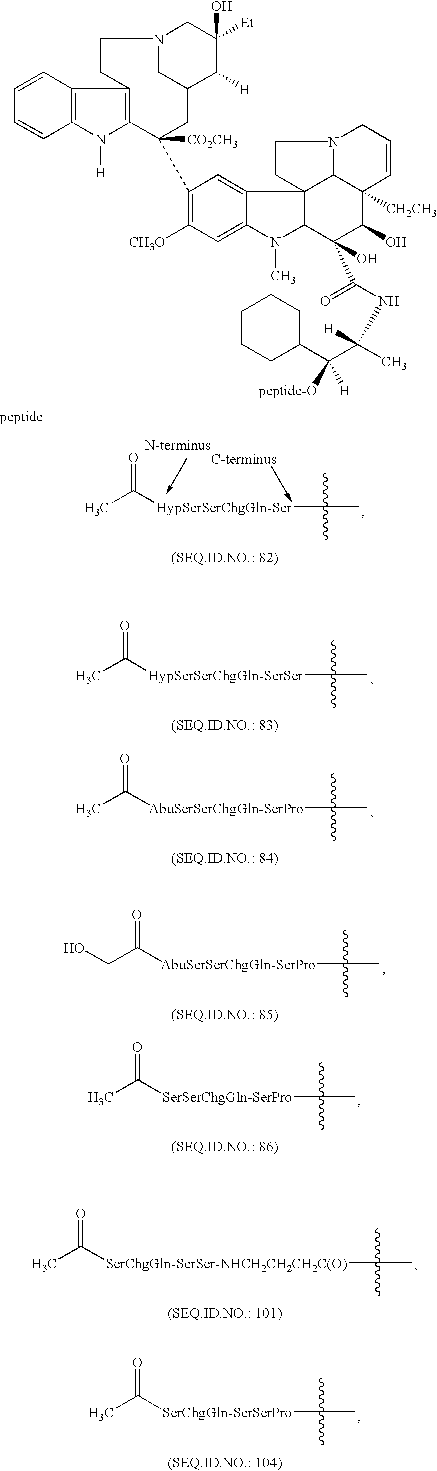

or the pharmaceutically acceptable salt thereof.

The oligopeptides, peptide subunits and peptide derivatives (also termed “peptides”) of the present invention can be synthesized from their constituent amino acids by conventional peptide synthesis techniques, preferably by solid-phase technology. The peptides are then purified by reverse-phase high performance liquid chromatography (HPLC).

Standard methods of peptide synthesis are disclosed, for example, in the following works: Schroeder et al., “The Peptides”, Vol. 1, Academic Press 1965; Bodansky et al., “Peptide Synthesis”, Interscience Publishers, 1966; McOmie (ed.) “Protective Groups in Organic Chemistry”, Plenum Press, 1973; Barany et al., “The Peptides: Analysis, Synthesis, Biology” 2, Chapter 1, Academic Press, 1980, and Stewart et al., “Solid Phase Peptide Synthesis”, Second Edition, Pierce Chemical Company, 1984. The teachings of these works are hereby incorporated by reference.

The suitably substituted cyclic amino acid having a hydrophilic substituent, which may be incorporated into the instant conjugates by standard peptide synthesis techniques, is itself either commercially available or is readily synthesized by techniques well known in the art or described herein. Thus syntheses of suitably substituted prolines are described in the following articles and references cited therein: J. Ezquerra et al., J. Org. Chem. 60: 2925-2930 (1995); P. Gill and W. D. Lubell, J. Org. Chem., 60:2658-2659 (1995); and M. W. Holladay et al., J. Med. Chem., 34:457-461 (1991). The teachings of these works are hereby incorporated by reference.

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, trifluoroacetic and the like.

The conjugates of the instant invention which comprise the oligopeptide containing the PSA cleavage site and a cytotoxic agent may similarly be synthesized by techniques well known in the medicinal chemistry art. For example, a free amine moiety on the cytotoxic agent may be covalently attached to the oligopeptide at the carboxyl terminus such that an amide bond is formed. Similarly, an amide bond may be formed by covalently coupling an amine moiety of the oligopeptide and a carboxyl moiety of the cytotoxic agent. For these purposes a reagent such as 2-(1H-benzotriazol-1-yl)-1,3,3-tetramethyluronium hexafluorophosphate (known as HBTU) and 1-hyroxybenzotriazole hydrate (known as HOBT), dicyclohexylcarbodiimide (DCC), N-ethyl-N-(3-dimethylamino-propyl)-carbodiimide (EDC), diphenylphosphorylazide (DPPA), benzotriazol- 1-yl-oxy-tris-(dimethylamino)phosphonium hexafluorophosphate (BOP) and the like, used in combination or singularly, may be utilized.

Furthermore, the instant conjugate may be formed by a non-peptidyl bond between the PSA cleavage site and a cytotoxic agent. For example, the cytotoxic agent may be covalently attached to the carboxyl terminus of the oligopeptide via a hydroxyl moiety on the cytotoxic agent, thereby forming an ester linkage. For this purpose a reagent such as a combination of HBTU and HOBT, a combination of BOP and imidazole, a combination of DCC and DMAP, and the like may be utilized. The carboxylic acid may also be activated by forming the nitrophenyl ester or the like and reacted in the presence of DBU (1,8-diazabicyclo[5,4,0]undec-7-ene.

One skilled in the art understands that in the synthesis of compounds of the invention, one may need to protect various reactive functionalities on the starting compounds and intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to Protective Groups in Organic Chemistry, McOmie, ed., Plenum Press, N.Y., N.Y. (1973); and, Protective Groups in Organic Synthesis, Greene, ed., John Wiley & Sons, N.Y., N.Y. (1981) for the teaching of protective groups which may be useful in the preparation of compounds of the present invention.

By way of example only, useful amino-protecting groups may include, for example, C₁-C₁₀alkanoyl groups such as formyl, acetyl, dichloroacetyl, propionyl, hexanoyl, 3,3-diethylhexanoyl, γ-chlorobutryl, and the like; C₁-C₁₀ alkoxycarbonyl and C₅-C₁₅aryloxycarbonyl groups such as tert-butoxycarbonyl, benzyloxycarbonyl, allyloxycarbonyl, 4-nitrobenzyloxycarbonyl, fluorenylmethyloxycarbonyl and cinnamoyloxycarbonyl; halo-(C₁-C₁₀)-alkoxycarbonyl such as 2,2,2-trichloroethoxycarbonyl; and C₁-C₁₅arylalkyl and alkenyl group such as benzyl, phenethyl, allyl, trityl, and the like. Other commonly used amino-protecting groups are those in the form of enamines prepared with β-keto-esters such as methyl or ethyl acetoacetate.

Useful carboxy-protecting groups may include, for example, C₁-C₁₀alkyl groups such as methyl, tert-butyl, decyl; halo-C₁-C₁₀alkyl such as 2,2,2-trichloroethyl, and 2-iodoethyl; C₅-C₁₅arylalkyl such as benzyl, 4-methoxybenzyl, 4-nitrobenzyl, triphenylmethyl, diphenylmethyl; C₁-C₁₀alkanoyloxymethyl such as acetoxymethyl, propionoxymethyl and the like; and groups such as phenacyl, 4-halophenacyl, allyl, dimethylallyl, tri-(C₁-C₃ alkyl)silyl, such as trimethylsilyl, β-p-toluenesulfonylethyl, β-p-nitrophenylthioethyl, 2,4,6-trimethylbenzyl, β-methylthioethyl, phthalimidomethyl, 2,4-dinitro-phenylsulphenyl, 2-nitrobenzhydryl and related groups.

Similarly, useful hydroxy protecting groups may include, for example, the formyl group, the chloroacetyl group, the benzyl group, the benzhydryl group, the trityl group, the 4-nitrobenzyl group, the trimethylsilyl group, the phenacyl group, the tert-butyl group, the methoxymethyl group, the tetrahydropyranyl group, and the like.

With respect to the preferred embodiment of an oligopeptide combined with vinblastine or desacetylvinblastine, the following Reaction Scheme illustrates the synthsis of the conjugates of the instant invention.

Reaction Scheme I illustrates preparation of conjugates of the oligopeptides of the instant invention and the vinca alkaloid cytotoxic agent vinblastine derivative wherein the attachment of vinblastine is via the linker to the C-terminus of the oligopeptide. Furthermore, Scheme I illustrates a synthesis of conjugates wherein the C-4-position hydroxy moiety is reacetylated following the addition of the linker unit. Applicants have discovered that the desacetyl vinblastine conjugate is also efficacious and may be prepared by eliminating the steps of reacting the intermediate with acetic anhydride, followed by deprotection of the amine. Addition of a single amino acid to the hydroxyalkylamine linker prior to the incorporation of the remaining peptide portion of the oligopeptide may be advantageous if the functionality of the amino acids that comprise the oligopeptide would compete with the nucleophillic hydroxyl moiety. Alternatively, if no such competing functional groups are present on the oligopeptide, the oligopeptide may be attached to the linker in a single reaction step.

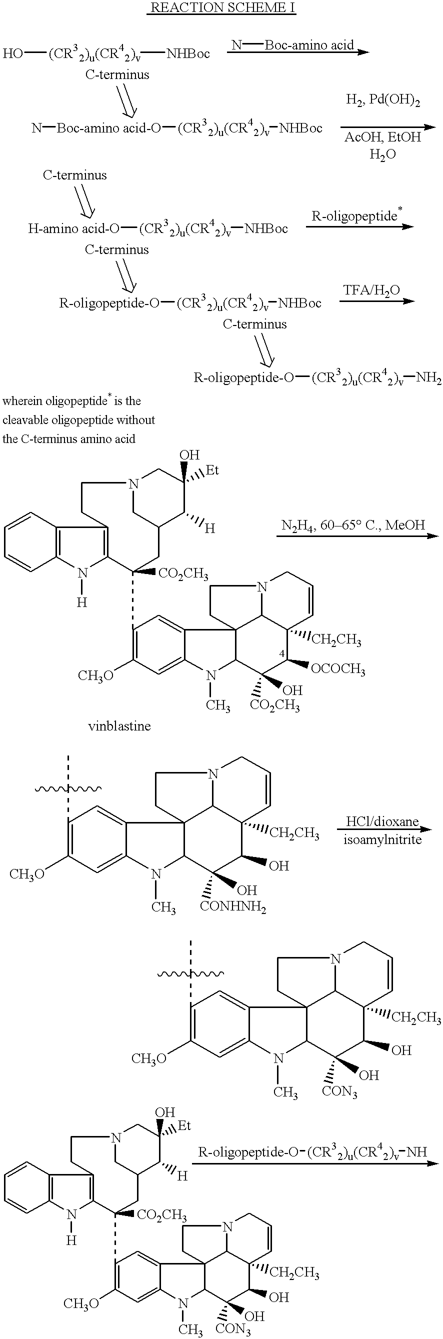

The novel cytotoxic agents of the instant invention which are derivatives of the vinca drug vinblastine may be described by the general formula II below:

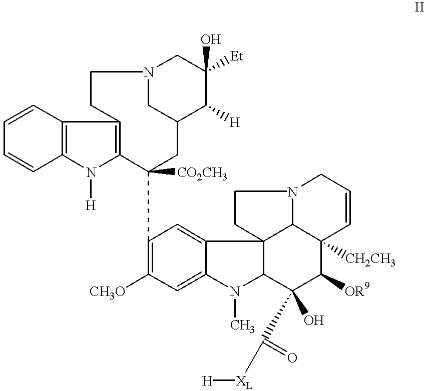

wherein:

X_{L} is selected from —NH—(CR³₂)ᵤ(CR⁴₂)ᵥ—O— and

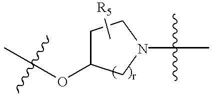

R³ and R⁴ are independently selected from: hydrogen, C₁-C₆-alkyl, hydroxylated C₃-C₈-cycloalkyl, polyhydroxylated C₃-C₈-cycloalkyl, hydroxylated aryl, polyhydroxylated aryl and aryl, or

one R³ and one R⁴ are combined to form a —(CH₂)_{w}—, which is unsubstituted or substituted with one or two substituents selected from OH and C₁-C₆alkyl; or

an R³ is combined with another R³ on the same carbon to form a —(CH₂)ₓ—; or

an R⁴ is combined with another R⁴ on the same carbon to form a —(CH₂)ₓ—;

R⁵ is selected from OH and C₁-C₆alkyl;

R⁹ is hydrogen, (C₁-C₃alkyl)—CO, or chlorosubstituted (C₁-C₃alkyl)—CO; and

r is 1, 2 or 3;

u and v are independently selected from: 0, 1, 2 or 3;

w is 2, 3 or 4;

x is 3, 4 or 5;

or the pharmaceutically acceptable salt or optical isomer thereof.

Preferably, u is 1 and v is 1.

Preferably, at least one R³ is selected from phenyl, cyclohexyl and cyclopentyl.

Preferably, at least one R⁴ is selected from phenyl, cyclohexyl, cyclopentyl and C₁-C₆alkyl.

The following compounds are specific examples of derivatives of the vinca drug vinblastine of the instant invention:

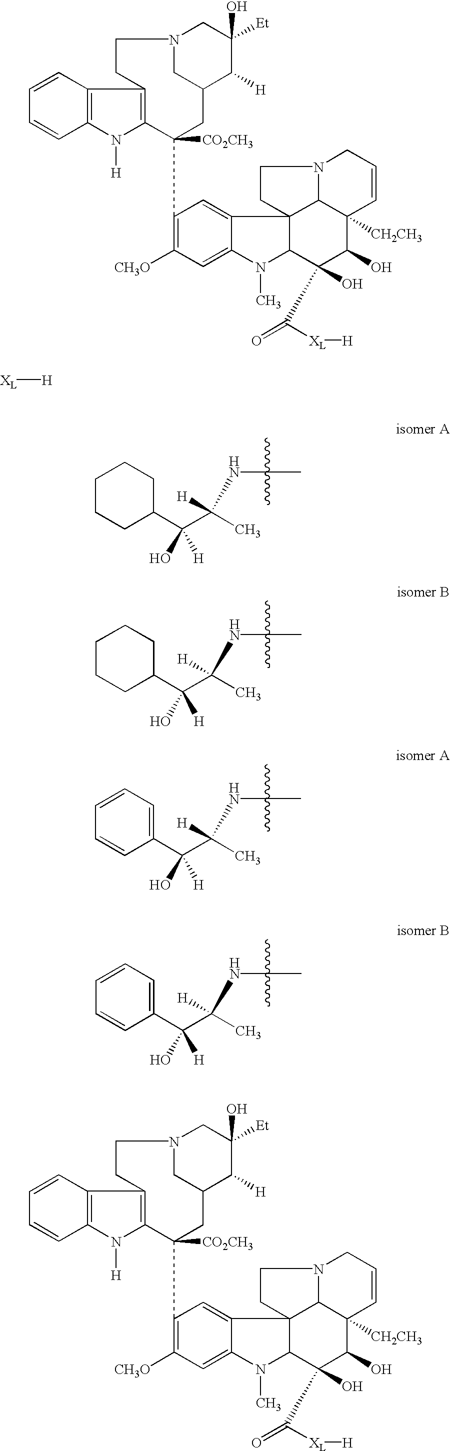

or the pharmaceutically acceptable salt or optical isomer thereof.

The pharmaceutically acceptable salts of the conjugates and novel cytotoxic agents of this invention include the conventional non-toxic salts of the compounds of this invention (also referred to as the compounds of the invention) as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The pharmaceutically acceptable salts of the compounds of this invention can be synthesized from the compounds of this invention which contain a basic moiety by conventional chemical methods. Generally, the salts are prepared either by ion exchange chromatography or by reacting the free base with stoichiometric amounts or with an excess of the desired salt-forming inorganic or organic acid in a suitable solvent or various combinations of solvents.

The oligopeptide-cytotoxic agent conjugates of the invention are administered to the patient in the form of a pharmaceutical composition which comprises a conjugate of of the instant invention and a pharmaceutically acceptable carrier, excipient or diluent therefor.

As used herein, “pharmaceutically acceptable” refers to those agents which are useful in the treatment or diagnosis of a warm-blooded animal including, for example, a human, equine, procine, bovine, murine, canine, feline, or other mammal, as well as an avian or other warm-blooded animal. The preferred mode of administration is parenterally, particularly by the intravenous, intramuscular, subcutaneous, intraperitoneal, or intralymphatic route. Such formulations can be prepared using carriers, diluents or excipients familiar to one skilled in the art. In this regard, See, e.g. Remington's Pharmaceutical Sciences, 16th ed., 1980, Mack Publishing Company, edited by Osol et al. Such compositions may include proteins, such as serum proteins, for example, human serum albumin, buffers or buffering substances such as phosphates, other salts, or electrolytes, and the like. Suitable diluents may include, for example, sterile water, isotonic saline, dilute aqueous dextrose, a polyhydric alcohol or mixtures of such alcohols, for example, glycerin, propylene glycol, polyethylene glycol and the like. The compositions may contain preservatives such as phenethyl alcohol, methyl and propyl parabens, thimerosal, and the like. If desired, the composition can include about 0.05 to about 0.20 percent by weight of an antioxidant such as sodium metabisulfite or sodium bisulfite.

As used herein, the term “composition” is intended to encompass a product comprising the specified ingredients in the specific amounts, as well as any product which results, directly or indirectly, from combination of the specific ingredients in the specified amounts.

For intravenous administration, the composition preferably will be prepared so that the amount administered to the patient will be from about 0.01 to about 1 g of the conjugate. Preferably, the amount administered will be in the range of about 0.2 g to about 1 g of the conjugate. The conjugates of the invention are effective over a wide dosage range depending on factors such as the disease state to be treated or the biological effect to be modified, the manner in which the conjugate is administered, the age, weight and condition of the patient as well as other factors to be determined by the treating physician. Thus, the amount administered to any given patient must be determined on an individual basis.

In utilizing the novel cytotoxic agents of formula II clinically, the clinical physician would administer them initially by the same route in the same vehicle and against the same types of tumors as for clinical use of leurocristine, vinblastine and vindesine. Differences in dosage levels would, of course, be based on the relative activity between the cytotoxic agents of formula II and the known vinca alkaloid drugs against the specific tumor type. The specific cancers that the cytotoxic agents of formula II may be useful against include, but are not limited to, haemotological tumors (such as chronic myologenis leukemia (CML), and acute lympoblastic leukemia (ALL)), prostate cancer and ovarian cancer.

The novel cytotoxic agents of formula II may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

For oral use of a cytotoxic agent according to this invention, the selected compound may be administered, for example, in the form of tablets or capsules, or as an aqueous solution or suspension. In the case of tablets for oral use, carriers which are commonly used include lactose and corn starch, and lubricating agents, such as magnesium stearate, are commonly added. For oral administration in capsule form, useful diluents include lactose and dried corn starch. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The cytotoxic agents of formula II may be administered at the rate of from 0.01 to 1 mg./kg. and preferably from 0.1 to 1 mg./kg. of the mammalian body weight once or twice a week or every two weeks depending on both the activity and the toxicity of the drug. An alternative method of arriving at a therapeutic dose is based on body surface area with a dose range of 0.1 to 10 mg./meter squared of mammalian body surface every 7 or 14 days.

The cytotoxic agents of the instant invention may also be co-administered with other well known therapeutic agents that are selected for their particular usefulness against the condition that is being treated. For example, the instant compounds may be useful in combination with known anti-cancer and cytotoxic agents.

One skilled in the art will appreciate that although specific reagents and reaction conditions are outlined in the following examples, modification can be made which are meant to be encompassed by the spirit and scope of the invention. The following preparations and examples, therefore, are provided to further illustrate the invention, and are not limiting.

### EXAMPLES

### Example 1

### Preparation of 4-des-Acetylvinblastine-23-(1S 2R)-(+)-2-Hydroxy-3-Cyclohexylisopropylamide Acetate Salt (1-3)

Step A 4-des-Acetylvinblastine-23-hydrazide (1-1)

A sample of 6.0 g (6.6 mmol) of vinblastine sulfate (Sigma V- 1377) was dissolved in 100 ml of 1:1 (v/v) absolute ethanol/anhydrous hydrazine, under N₂, and the solution was heated in an oil bath at 60-65° C. for 23 hr. Upon cooling, the solution was evaporated to a thick paste, which was partitioned between 350 ml of CH₂Cl₂ and 200 ml of 2.5% aq. NaHCO₃. The aqueous layer was extracted with 2 100-ml portions of CH₂Cl₂, and each of the 3 organic layers in turn was washed with 100 ml each of H2O (2X) and saturated NaCl (1X). The combined organic layers were dried over anhydrous Na₂SO₄, and the solvent was removed in vacuo to yield, after drying 6 hr in vacuo, the title compound as a white crystalline solid (1-1).

Step B: (1S , 2R)-(+)-2-Hydroxy-3-Cyclohexylisopropylamine (HCAP) (1-2)

A solution of 2.00 g of (1S, 2R)-(+)-Norephedrine in 50 ml acetic acid/10 ml H₂O was hydrogenated at 62 psi on a Parr apparatus over 500 mg of Ir black catalyst. After 24 h, a second portion of catalyst was added and the reaction continued for a second 24 h interval. The reaction was filtered through a Celite pad, and the filtrate concentrated in vacuo to give a tan foam (1-2). FABMS: 158

Step C: Preparation of 4-des- Acetylvinblastine-23-(1S, 2R)-(+)-2-Hydroxy-3-Cyclohexylisopropylamide (HCAP-(dAc)vinblastine (1-3)

A solution of 0.922 of 4-des-acetylvinblastine-23-hydrazide (1.2 mmol) in 20 ml DMF cooled to −15° C. under Argon, was converted to the azide in situ by acidification with 4 M HCl in dioxane to pH<1.5 (moistened 0-2.5 range paper), followed by addition of 0.21 ml (1.3 equiv) of isoamyl nitrite and stirring for 1 hr at 10-15° C. The pH was brought to 7 by the addition of DIEA, and a slurry of 0.37 g (2.4 mmol) of HCAP (1-2) product from step B was then added, and the reaction was stirred at 0° C. for 10 hrs, at which point coupling was complete, as monitored by analytical HPLC (A=0.1% TFA/H₂O; B=0.1% TFA/CH₃CN). The reaction was concentrated to a small volume in vacuo, then purified by preparatory HPLC on a 15 μM, 100 A, Delta-Pak C18 column with 0.1% trifluoroacetic acid-aqueous acetonitrile solvent systems using 95-50% A, 60 min linear gradient. Homogeneous fractions were pooled and concentrated in vacuo, followed by freeze-drying to give the title compound as the TFA salt (1-3).

FABMS: 893

HPLC: 99% pure @214 nm, retention time=18.42 min, (Vydac C₁₈, gradient of 95% A/B to 5% A/B over 30 min, A=0.1% TFA-H₂O, B=0.1 % TFA-CH₃CN)

Table 3 shows the compound described in Example 1 and other vinca drug derivatives that were prepared by the procedure described in Example 1, but utilizing the appropriate amine in Step C. Unless otherwise indicated, the trifluoroacetate salt of the conjugate was prepared and tested.

TABLE 3

| | LNCaP Cell Kill in 72 |
|---|---|
| | HRS |
| Cytotoxic Agent | EC5O (μM) |
| VINBLASTINE | 0.5 (T24 = <0.08) |
| (dAc)-VINBLASTINE | 0.3 (Colo3209DM = 0.5) |
| L-phenylalaninol-(dAc)-VIN | 0.5 (Colo320DM = 3.5) |
| L-isoleucinol-(dAc)-VIN | 0.9 (Colo320DM = 1.7) |
| L-Valinol-(dAc)-VIN | 0.4 (Colo320DM = 0.8 |
| L-leucinol-(dAc)-VIN | 0.7 (Colo320DM = 2.0) |
| Serinol-(dAc)-VIN | 0.8 (Colo320DM = 8.3) |
| 2-Aminobutanol-(dAc)-VIN | 2.9 (Colo320DM = 7.1) |
| L-cyclolhexyl-alaninol-(dAc)-VIN | 1.0 (Colo320DM = 2.0) |
| L-cyclopropyl-alainine-OEt-(dAc)-VIN | 1.4 (Colo320DM = 1.0) |
| Phenylglyinol-(dAc)-VIN | 0.7 (Colo320DM = 4.8) |
| 1,2-diPhenylethanolamino-(dAc)-VIN | 2.2 (Colo320DM = 8.9) |
| 2-hydroxylpropylamino-(dAc)-VIN | 1.2 (Colo320DM = 2.9) |
| 3-hydroxylpyrrolidine-(dAc)-VIN | 0.2 (Colo320DM = 1.5) |
| 4-hydroxylpiperidine-(dAc)-VIN | 0.2 (Colo320DM = 0.8) |
| (trans-2-hydroxyl)cyclohexylamine- | 0.1 (Colo320DM = 0.2) |
| (dAc)-VIN, Isomer A | |
| (trans-2-hydroxyl)cyclohexylamino- | 0.8 (Colo320DM = 0.8) |
| (dAc)-VIN, Isomer B | |
| 1-hydroxylcyclohexylmethyamino- | 0.5 (Colo320DM = 15.8) |
| (dAc)-VIN | |
| norephedrine-(dAc)-VIN, isomer A | 3.0 (Colo320DM = 3.0) |
| norephedrine-(dAc)-VIN, isomer B | 0.2 (Colo320DM = 0.4) |
| 3-methoxy-norephedrine-(dAc)-VIN | 1.8 (Colo320DM = 5.1) |
| 3-hydroxyl-piperidine-(dAc)-VIN, | 0.5 (Colo320DM = 0.5) |
| isomer A | |
| 3-hydroxyl-piperidine-(dAc)-VIN, | 0.5 (Colo320DM = 0.5) |
| isomer B | |
| tryptophanol-(dAc)-VIN | 0.6 (Colo320DM = 2.9) |
| (3-cyclohexyl-3-hydroxyl-2- | 0.3 (Colo320DM = 0.5) |
| propylaminol)-(dAc)-VIN isomer A | |
| (3-cyclohexyl-3-hydroxyl-2- | 1.2 (Colo320DM = 0.8) |
| propylaminol)-(dAc)-VIN isomer B | |

wherein:

(dAc)-VIN is

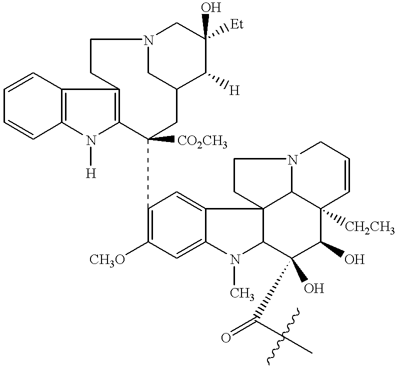

wherein the attachment to the rest of the compound is through the nitrogen of the hydroxyalkylamine.

### Example 2

### Preparation of 4-des-Acetylvinblastine-23-(N-Acetyl-4-trans-L-Hyp-Ser-Ser-Chg-Gln-Ser-HCAP) Amide Acetate Salt (2-7)

Step A: N-Acetyl-4-trans-L-Hyp-Ser-Ser-Chg-Gln-OH (2-1) (SEQ.ID.NO. 87)

Starting with 0.5 mmole (0.80 g) of Fmoc-Gln(Trt)-Wang resin, the protected peptide was synthesized on a ABI model 430A peptide synthesizer. The protocol used a 4-fold excess (2.0 mmol) of each of the following protected amino acids: Fmoc-Ser(tBu)-OH, Fmoc-Chg-OH, Fmoc-4-trans-Hyp(tBu)-OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95% TFA: 2.5% H₂O: 2.5% Triisopropylsilane (20 ml) for 2 hr at r.t. under argon. After evaporation of the TFA, the residue was washed with ether, filtered and dried to give crude peptide which was purified by preparatory HPLC on a Delta-Pak C18 column with 0.1% trifluoroacetic acid-aqueous acetonitrile solvent systems using 100-70% A, 60 min linear gradient. Fractions containing product of at least 99% (HPLC) purity were combined to give the title compound.

FABMS: 615.3

Peptide Content: 1.03 nmole/mg.

HPLC: 99% pure @214 nm, retention time=10.16 min, (Vydac C₁₈, gradient of 95% A/B to 50% A/B over 30 min, A=0.1% TFA-H₂O, B=0.1% TFA-CH₃CN)

In a similar manner the following compound was prepared:

N-hydroxyacetyl-Abu-Ser-Ser-Chg-Gln-Ser-OH (3-1) (SEQ.ID.NO. 88)

Step B: N-Boc-(1S, 2R)-(+)-Norephedrine (2-2)

A solution of 1.51 g (10 mmol) of (1S, 2R)-(+)-Norephedrine in a mixture of 1,4 dioxane (20 ml), water (10 ml) and 1 N NaOH (10 ml) was stirred and cooled in an ice-water bath. Di-(t-butyl) dicarbonate (2.4 g, 11 mmol) was added in portions over approx. 20 min. The reaction was stirred in the cold for 2 hrs., then at room temp. for an additional 1 h. The solution was concentrated to remove most of the dioxane, cooled in an ice bath and covered with a layer of ethyl acetate (30 ml) and acidified to pH 2 with 1 N KHSO₄. The aqueous phase was extracted 2x with EtOAc. The combined extracts were washed with water, brine and were concentrated and dried to provide the desired product as a white crystalline solid (2-2).

FABMS: 252

Step C: N-Boc-HCAP (2-3)

A solution of 2.38 g of N-Boc-(1S,2R)-(+)-Norephedrine (2-2) in 50 ml acetic acid/10 ml H₂O was hydrogenated at 60 psi on a Parr apparatus over 500 mg of Ir black catalyst for 24 hrs. The reaction was filtered through a Celite pad, and the filtrate concentrated in vacuo to give a tan foam (2-3). FABMS: 258.2

Step D: N-Benzyloxycarbonyl-Ser-N-t-Boc-HCAP ester (2-4)

A solution of 1.95 g (6.6 mmol) of N-Z-Ser(tBu)-OH,

1.54 g (6.0 mmol) of N-Boc-HCAP (2-3), 1.26 g (6.6 mmol) of EDC, and 146 mg (1.2 mmol) of DMAP in 30 ml of anh. CH2Cl2 was treated and the resulting solution stirred at room temp. in an N₂ atmosphere for 12 h. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product (2-4).

In a similar manner the following compound was prepared:

N-Benzyloxycarbonyl-Pro-N-t-Boc-HCAP ester (3-2) by coupling of N-Z-Pro-OH with N-Boc-HCAP alcohol (2-3)

Step E: H-Ser(tBu)-N-t-Boc-HCAP ester (2-5)

A 2.0 g of (2-4) in a solution of 90 ml EtOH, 20 ml water, and 10 ml acetic acid was hydrogenated on a Parr apparatus at 50 psi over 200 mg of Pd(OH)₂ catalyst for 3 h. The reaction was filtered through a Celite pad, and the concentrated to small volume in vacuo, then purified by preparatory HPLC on a Delta-Pak C18 column with 0.1% trifluoroacetic acid -aqueous acetonitrile solvent systems using 95-50% A, 60 min linear gradient. Fractions containing product of at least 99% (HPLC) purity were combined to give the intermediate (2-5).

FABMS: 401.3

In a similar manner the following compound was prepared:

H-Pro-N-t-Boc-HCAP ester (3-3)

by hydrogenation of N-Benzyloxycarbonyl-Pro-N-t-Boc-HCAP ester (3-2)

Step F: N-Acetyl-4-trans-L-Hyp-Ser-Ser-Chg-Gln-Ser-HCAP amine (2-6) (SEQ.ID.NO. 82)

A solution of 614 mg (1.0 mmol) of N-Acetyl-4-trans-L Hyp-Ser-Ser-Chg-Gln-OH (2-1), 400 mg (1.0 mmol) of H-Ser(tBu)-N-t-Boc-HCAP ester (2-5), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 h. The solvent was removed in vacuo, the residue was washed with ether and dried. The crude product was treated with 95% TFA : 5% H₂O (20 ml) for 2 hr at r.t. under argon. After evaporation of the TFA, the residue was purified by preparatory HPLC on a Delta-Pak C18 column with 0.1% trifluoroacetic acid-aqueous acetonitrile solvent systems using 95-50% A, 60 min linear gradient. Fractions containing product of at least 99% (HPLC) purity were combined to give the intermediate compound (2-6).

FABMS: 841.8

Peptide Content: 863.39 NMole/mg.

HPLC: 99% pure @214 nm, retention time=13.7 min, (Vydac C18, gradient of 95% A/B to 5% A/B over 30 min, A=0.1% TFA-H₂O, B=0.1 % TFA-CH₃CN)

In a similar manner the following compound was prepared:

N-Hydroxyacetyl-Abu-Ser-Ser-Chg-Gln-Ser-Pro-HCAP amine (3-4) (SEQ.ID.NO. 89)

by coupling of N-Hydroxyacetyl-Abu-Ser-Ser-Chg-Gln-Ser-OH (3-1) with H-Pro-N-t-Boc-HCAP ester (3-3) followed by deprotection.

Step G: 4-des-Acetylvinblastine-23-(N-Ac-4-trans-L-Hyp-Ser-Ser-Chg-Gln-Ser-HCAP) amide acetate salt (2-7)

A solution of 0.461 of 4-des-acetylvinblastine-23-hydrazide (0.6 mmol) in 10 ml DMF cooled to −15° C. under Argon, was converted to the azide in situ by acidification with 4 M HCl in dioxane to pH<1.5 (moistened 0-2.5 range paper), followed by addition of 0.105 ml (1.3 equiv) of isoamyl nitrite and stirring for 1 hr at 10-15° C. The pH was brought to 7 by the addition of DIEA, and 555 mg (0.66 mmol) of amine derivative (2-6) from step F was then added, and the reaction was stirred at 0° C. for 24 hrs, and purified by preparatory HPLC on a 15 μM, 100 A, Delta-Pak C18 column with 0.1% trifluoroacetic acid -aqueous acetonitrile solvent systems using 95-50% A, 60 min linear gradient. Homogeneous fractions were pooled and concentrated in vacuo, followed by freeze-drying to give the title compound as the TFA salt which was converted to 420 mg HOAc salt by AG 4×4 resin (100-200 mesh, free base form, BIO-RAD) (2-7)

ES⁺: 1576.7

Peptide Content: 461.81 NMole/mg.

Ser 3.04; Hyp 1.07; Chg 1.02; Glu 1.00

HPLC: 99% pure @214 nm, retention time=18.31 min, (Vydac C18, gradient of 95% A/B to 5% A/B over 30 min, A=0.1% TFA-H₂O, B=0.1% TFA-CH₃CN)

In a similar manner the following compound was prepared:

4-des-Acetylvinblastine-23-(N-hydroxyacetyl-Abu-Ser-Ser-Chg-Gln-Ser-Pro-HCAP) amide (3-5)

by coupling 4-des-Acetylvinblastine-23-hydrazide (1-1) with OH-Acetyl-Abu-Ser-Ser-Chg-Gln-Ser-Pro-HCAP amine (3-4) 4-des-Acetylvinblastine-23-(N-hydroxyl-Ac-Abu-Ser-Ser-Chg-Gln-Ser-HCAP) amide acetate salt (3-5)

ES⁺: 1661.9

Peptide Content: 499.87 NMole/mg.

Ser 2.98; Abu 1.01; Chg 1.02; Glu 1.00; Pro 0.98

HPLC: 99% pure @214 nm, retention time=18.83 min, (Vydac C18, gradient of 95% A/B to 5% A/B over 30 min, A=0.1% TFA-H₂O, B=0.1% TFA-CH₃CN)

### Example 2A

### Preparation of 4-des-Acetylvinblastine-23-(N-Acetyl-Ser-Chg-Gln-Ser-Ser-Pro-HCAP) Amide Acetate Salt (2A-7)

Step A: N-Acetyl-Ser-Chg-Gln-Ser-Ser-OH (2A-1)

Starting with 0.5 mmole (0.80 g) of Fmoc-Ser(tBu)-Wang resin, the protected peptide was synthesized on a ABI model 430 A peptide synthesizer. The protocol used a 4-fold excess (2.0 mmol) of each of the following protected amino acids: Fmoc-Ser(tBu)-OH, Fmoc-Gln-OH, Fmoc-Chg-OH, Fmoc-Ser(tBu)-OH and acetic acid (2 couplings). During each coupling cycle Fmoc protection was removed using 20% piperidine in DMF. Coupling was achieved using DCC and HOBt activation in N- methyl-2-pyrrolidinone. At the completion of the synthesis, the peptide resin was dried. 1.3 g peptide-resin was treated with 95%TFA :2.5% H₂O:2.5% Triisopropylsilane (20 ml) for 2 hr at r.t. under argon. After evaporation of the TFA, the residue was washed with ether, filtered and dried to give crude peptide which was purified by preparatory HPLC on a Delta-Pak C18 column with 0.1% trifluoroacetic acid-aqueous acetonitrile solvent systems using 100-70% A, 60 min linear gradient. Fractions containing product of at least 99% (HPLC) purity were combined to give the title compound.

FABMS: 589.5

Peptide Content: 1.01 NMole/mg.

HPLC: 99% pure @214 nm, retention time=10.7 min, (Vydac C18, gradient of 95% A/B to 50% A/B over 30 min, A=0.1% TFA-H₂O, B=0.1% TFA-CH₃CN)

Step B: N-Boc-(1S, 2R)-(+)-Norephedrine (2A-2)

A solution of 1.51 g (10 mmol) of (1S, 2R)-(+)-Norephedrine in a mixture of 1,4 dioxane (20 ml), water (10 ml) and 1 N NaOH (10 ml) is stirred and cooled in an ice-water bath. Di-(t-butyl) dicarbonate (2.4 g, 11 mmol) was added in portions over approx. 20 min. The reaction was stirred in the cold for 2 hrs., then at room temp. for an additional 1 h. The solution was concentrated to remove most of the dioxane, cooled in an ice bath and covered with a layer of ethyl acetate (30 ml) and acidified to pH 2 with 1 N KHSO₄. The aqueous phase was extracted 2× with EtOAc. The combined extracts were washed with water, brine and were concentrated and dried to provide the desired product as a white crystalline solid. FABMS: 252

Step C: N-Boc-HCAP (2A-3)

A solution of 2.38 g of N-Boc-(1S, 2R)-(+)-Norephedrine (2A-2) in 50 ml acetic acid/10 ml H₂O was hydrogenated at 60 psi on a Parr apparatus over 500 mg of Ir black catalyst for 24 hrs. The reaction was filtered through a Celite pad, and the filtrate concentrated in vacuo to give a tan foam. FABMS: 258.2

Step D: N-Benzyloxycarbonyl-Pro-N-t-Boc-HCAP ester (2A-4)

A solution of 1.62 g (6.6 mmol) of N-Z-Pro-OH, 1.54 g (6.0 mmol) of N-Boc-HCAP (2A-3), 1.26 g (6.6 mmol) of EDC, and 146 mg (1.2 mmol) of DMAP in 30 ml of anh. CH2C12 was treated and the resulting solution stirred at room temp. in an N₂ atmosphere for 12 h. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product.

Step E: H-Pro-N-t-Boc-HCAP ester (2A-5)

A 2.0 g of (2A-4) in a solution of 90 ml EtOH, 20 ml water, and 10 ml acetic acid was hydrogenated on a Parr apparatus at 50 psi over 200 mg of Pd(OH)₂ catalyst for 3 h. The reaction was filtered through a Celite pad, and the concentrated to small volume in vacuo, then purified by preparatory HPLC on a Delta-Pak C18 column with 0.1% trifluoroacetic acid -aqueous acetonitrile solvent systems using 95-50% A, 60 min linear gradient. Fractions containing product of at least 99% (HPLC) purity were combined to give the title compound (2A-5).

FABMS: 356.3

Step F: N-Acetyl -Ser-Chg-Gln-Ser-Ser-Pro-HCAP amine (2A-6)

A solution of 589 mg (1.0 mmol) of N-Acetyl-Ser-Chg-Gln-Ser-Ser-OH (2-1), 356 mg (1.0 mmol) of H-Pro-N-t-Boc-HCAP ester (2A-5), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 h. The solvent was removed in vacuo, the residue was washed with ether and dried. The crude product was treated with 95% TFA : 5% H₂O (20 ml) for 2 hr at r.t. under argon. After evaporation of the TFA, the residue was purified by preparatory HPLC on a Delta-Pak C18 column with 0.1% trifluoroacetic acid-aqueous acetonitrile solvent systems using 95-50% A, 60 min linear gradient. Fractions containing product of at least 99% (HPLC) purity were combined to give the title compound (2-6).

FABMS: 825.5

Peptide Content: 893.6 NMole/mg.

HPLC: 99% pure @214 nm, retention time=15.2 min, (Vydac C18, gradient of 95% A/B to 5% A/B over 30 min, A=0.1% TFA-H₂O, B=0.1% TFA-CH₃CN)

Step G: 4-des-Acetylvinblastine-23-(N-Ac-Ser-Chg-Gln-Ser-Ser-Pro-HCAP) amide acetate salt (2A-7)

A solution of 0.461 of 4-des-acetylvinblastine-23-hydrazide (0.6 mmol) in 10 ml DMF cooled to −15° C. under Argon, was converted to the azide in situ by acidification with 4 M HCl in dioxane to pH<1.5 (moistened 0-2.5 range paper), followed by addition of 0.105 ml (1.3 equiv) of isoamyl nitrite and stirring for 1 hr at 10-15° C. The pH was brought to 7 by the addition of DIEA, and 545 mg (0.66 mmol) of amine derivative (2A-6) from step F was then added, and the reaction was stirred at 0° C. for 24 hrs, and purified by preparatory HPLC on a 15 μM, 100 A, Delta-Pak C18 column with 0.1% trifluoroacetic acid -aqueous acetonitrile solvent systems using 95-50% A, 60 min linear gradient. Homogeneous fractions were pooled and concentrated in vacuo, followed by freeze-drying to give the title compound as the TFA salt which was converted to title compound by AG 4×4 resin (100-200 mesh, free base form, BIO-RAD) (2A-7)

ES: 1560.9

Peptide Content: 586.8 NMole/mg.

Ser 3.04; Chg 1.01; Glu 1.00; Pro 0.97

HPLC: 99% pure @214 nm, retention time=13.4 min, (Vydac C18, gradient of 95% A/B to 5% A/B over 30 min, A=0.1% TFA-H₂O, B=0.1% TFA-CH₃CN)

Table 4 shows the compounds described in Examples 2 and 2A and other peptide-vinca drug conjugates that were prepared by the procedures described in Examples 2 and 2A, but utilizing the appropriate amino acid residues and blocking group acylation. Unless otherwise indicated, the acetate salt of the conjugate was prepared and tested.

TABLE 4

| | | Time to 50% |
|---|---|---|
| | | Substrate |
| | | Cleavage |
| SEQ. | | by York |
| ID. NO. | PEPTIDE-VIN CONJUGATE | PSA (Min) |
| 90 | Ac-(4-trans-L-Hyp)SSChgQ-SPheol-(dAc)-VIN | 25 |
| 91 | Ac-4-trans-L-HypSSChgQS-cyclopropylalaninol- | 45 |
| | (dAc)-VIN | |
| 92 | Ac-4-trans-L-HypSSChgQS-cyclohexylalaninol- | 10 |
| | (dAc)-VIN | |
| 93 | Ac-4-trans-L-HypSSChgQS-valinol-(dAc)-VIN | 80 |
| 82 | Ac-4-trans-L-HypSSChgQS-(HCAP)-(dAc)-VIN | 12 |
| | TFA salt | |
| 82 | Ac-4-trans-L-HypSSChgQS-(HCAP)-(dAc)-VIN | 15 |
| | Acetate salt | |
| 82 | Ac-4-trans-L-HypSSChgQS-O-3(R)pyrrolidine- | 14 (n = 2) |
| | (HCAP)-(dAc)-VIN | |
| 83 | Ac-4-trans-L-Hyp-SSChgQ-SS-(HCAP)-(dAc)- | 17 |
| | VIN | |
| 85 | N-hydroxyacetyl-AbuSSChgQ-SP-(HCAP)- | 11 |
| | (dAc)-VIN | |
| 86 | Ac-SSChgQ-SP-(HCAP)-(dAc)-VIN | 30 |
| 84 | Ac-AbuSSChgQ-SP-(HCAP)-(dAc)-VIN | 18 |
| 94 | Ac-SChgQ-SP-(HCAP)-(dAc)-VIN | 13 |
| 95 | Ac-AbuSChgQ-SP-(HCAP)-(dAc)-VIN | 17 (n = 2) |
| 96 | Ac-SChgQSS-Sar-(HCAP)-dAc-VIN | 13 |
| 97 | Ac-SChgQS-Abu-(HCAP)-VIN | 60 |
| 98 | Ac-SChgQ-SS(4-trans-L-Hyp)-(HCAP)-dAc-VIN | 7 |
| 99 | Ac-SChgQSS(PIP)-(HCAP)-dAc-VIN | 22 |
| 100 | Ac-SChgQSS(HCAP)-dAc-VIN | 12 |
| 101 | Ac-SChgQSS-gammaAbu-(HCAP)-dAc-VIN | 12 |
| 102 | Ac-4-trans-L-HypSSChgQSP(HCAP)-VIN | 8 |
| 103 | Ac-SSChgQ-SSP-(HCAP)-dAc-VIN | 8 |
| 104 | Ac-SChgQ-SSP-(HCAP)-VIN | 8 |
| 105 | Ac-AbuSSChgQ-S-(HCAP)-VIN | 1 HOUR = |
| | | 28% |

4-trans-L-Hyp is trans-4-hydroxy-L-proline.
Pheol is phenylalaninol
Sar is sarcosine
PIP is pipecolinic acid
Abu is 2-aminobutyric acid
gammaAbu is 4-aminobutyric acid
(dAc)-VIN is as described for Table 3.
(HCAP)-(dAc)-VIN is

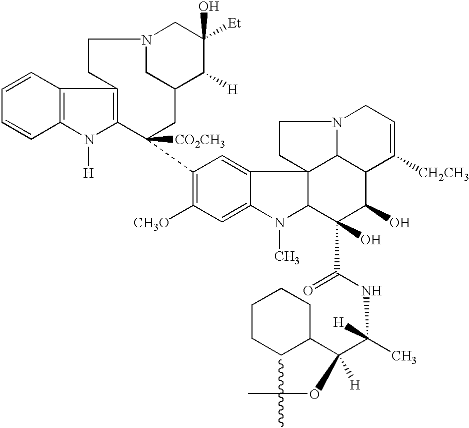

when n > 1; value is an average

### Example 3

### Assessment of the Recognition of Oligopeptide-Vinca Drug Conjugates by Free PSA

The conjugates prepared as described in Example 3 were individually dissolved in PSA digestion buffer (50 mM tris(hydroxymethyl)-aminomethane pH7.4, 140 mM NaCl) and the solution added to PSA at a molar ration of 100 to 1. Alternatively, the PSA digestion buffer utilized is 50 mM tris(hydroxymethyl)-aminomethane pH7.4, 140 mM NaCl. The reaction is quenched after various reaction times by the addition of trifluoroacetic acid (TFA) to a final 1% (volume/volume). Alternatively the reaction is quenched with 10 mM ZnCl2. The quenched reaction was analyzed by HPLC on a reversed-phase C18 column using an aqueous 0.1% TFA/acetonitrile gradient. The results of the assessment are shown in Table 4. Table 4 shows the amount of time (in minutes) required for 50% cleavage of the noted oligopeptide-cytotoxic agent conjugates with enzymatically active free PSA. Unless otherwise indicated, the acetate salt of the conjugate was tested.

### Example 4

### In vitro Assay of Cytotoxicity of Peptidyl Derivatives of Vinca Drugs

The cytotoxicities of the vinca alkaloid derivatives, prepared as described in Example 1, and the cleaveable oligopeptide-vinca drug conjugates, prepared as described in Examples 2 and 2A, against a line of cells which is known to be killed by unmodified vinca drug was assessed with an Alamar Blue assay. Specifically, cell cultures of LNCap prostate tumor cells, Colo320DM cells (also designated C320), T24 bladder carcinoma cells or T47D breast carcinoma cells in 96 well plates was diluted with medium containing various concentrations of a given conjugate (final plate well volume of 200 μl). The cells were incubated for 3 days at 37° C., 20 μl of Alamar Blue is added to the assay well. The cells were further incubated and the assay plates were read on a EL-310 ELISA reader at the dual wavelengths of 570 and 600 nm at 4 and 7 hours after addition of Alamar Blue. Relative percentage viability at the various concentration of conjugate tested was then calculated versus control (no cytotoxic agent or conjugate) cultures. Results of this assay are shown in Tables 3 and 5. Unless otherwise indicated, the TFA salt of the cytotoxic agent and the acetate salt of the conjugate were tested.

TABLE 5

| | | LNCaP Cell Kill in |
|---|---|---|
| SEQ. | | 72 HRS |
| ID. NO | PEPTIDE-VIN CONJUGATE | EC5O (μM) |
| | VINBLASTINE | 0.5 (T24 = <0.08) |
| 90 | Ac-(4-trans-L-Hyp)SSChgQ-SPheol- | 1.3 (Colo320DM = |
| | (dAc)-VIN | 2.3) PS, labile in |
| | | mouse serum |
| 91 | Ac-4-trans-L-HypSSChgQS- | 1.5 (Colo320DM = |
| | cyclopropylalaninol-(dAc)-VIN | 7.5) ester bond |
| | | lability |
| 92 | Ac-4-trans-L-HypSSChgQS-cyclohexyl- | 0.3 (Colo320DM = |
| | alaninol-(dAc)-VIN | 2.6) |
| 93 | Ac-4-trans-L-HypSSChgQS-valinol- | NA |
| | (dAc)-VIN | |
| 82 | Ac-4-trans-L-HypSSChgQS-(HCAP)- | 2.0 (Colo320DM = |
| | (dAc)-VIN TFA salt | 4.1) |
| 82 | Ac-4-trans-L-HypSSChgQS-(HCAP)- | 3.4 (Colo320DM = |
| | (dAc)-VIN Acetate salt | 4.6) n = 2 |
| 82 | Ac-4-trans-L-HypSSChgQS-O-3(R)pyrrol- | 1.9 (Colo320DM = |
| | idine-(HCAP)-(dAc)-VIN | 30) |
| 83 | Ac-4-trans-L-HypSSChgQ-SS-(HCAP)- | 2.0 (Colo320DM = |
| | (dAc)-VIN | 5.0) |
| 85 | (2-OH)Ac-AbuSSChgQ-SP-(HCAP)- | 2.0 (Colo320DM = |
| | (dAc)-VIN | 12.6) |
| 86 | Ac-SSChgQ-SP-(HCAP)-(dAc)-VIN | 10.2 (Colo320DM = |
| | | 29.5) |
| 84 | Ac-AbuSSChgQ-SP-(HCAP)-(dAc)-VIN | 2.0 (Colo320DM = |
| | | 15.7) |
| 94 | Ac-SChgQ-SP-(HCAP)-(dAc)-VIN | 7.8 (Colo320DM = |
| | | 15.7) |
| 95 | Ac-AbuSChgQ-SP-(HCAP)-(dAc)-VIN | 4.1 (Colo320DM = |
| | | 15.7) |
| 96 | Ac-SChgQSS-Sar-(HCAP)-dAc-VIN | |
| 97 | Ac-SChgQS-Abu-(HCAP)-VIN | 0.8 (Colo320DM = |
| | | 2.0) |
| 98 | Ac-SChgQ-SS(4-trans-L-Hyp) -(HCAP)- | 5.9 (Colo320DM = |
| | dAc-VIN | 10.4) |
| 99 | Ac-SChgQSS(PIP)-(HCAP)-dAc-VIN | |
| 100 | Ac-SChgQSS(HCAP)-dAc-VIN | 1.4 (Colo320DM = |
| | | 1.4) |
| 101 | Ac-SChgQSS-gammaAbu-(HCAP)-dAc- | 2.3 (Colo320DM = |
| | VIN | 4.3) |
| 102 | Ac-4-trans-L-HypSSChgQSP(HCAP)- | 5.5 (Colo320DM = |
| | VIN | 15.6) |
| 103 | Ac-SSChgQ-SSP-(HCAP)-dAc-VIN | 2.6 (Colo320DM = |
| | | 6.3) |
| 104 | Ac-SChgQ-SSP-(HCAP)-VIN | 7.8 (Colo320DM = |
| | | 15.7) |
| 105 | Ac-AbuSSChgQ-S-(HCAP-VIN | 6.1 (Colo320DM = |
| | | 7.8 |

4-trans-L-Hyp is trans-4-hydroxy-L-proline.
(dAc)-VIN is as described for Table 3.
(HCAP)-(dAc)-VIN, Sar, Abu, gammaAbu and PIP are as described for Table 4.

### Example 5

### In vivo Efficacy of Peptidyl -Cytotoxic Agent Conjugates

LNCaP.FGC or C320 cells are trypsinized, resuspended in the growth medium and centifuged for 6 mins. at 200×g. The cells are resuspended in serum-free a-MEM and counted. The appropriate volume of this solution containing the desired number of cells is then transferred to a conical centrifuge tube, centrifuged as before and resuspended in the appropriate volume of a cold 1:1 mixture of a-MEM-Matrigel. The suspension is kept on ice until the animals are inoculated.

Harlan Sprague Dawley male nude mice (10-12 weeks old) are restrained without anesthesia and are inoculated with 0.5 mL of cell suspension on the left flank by subcutaneous injection using a 22 G needle. Mice are either given approximately 5×105 DuPRO cells or 1.5×107 LNCaP.FGC cells.

Following inoculation with the tumor cells the mice are treated under one of two protocols:

Protocol A:

One day after cell inoculation the animals are dosed with a 0.1-0.5 mL volume of test conjugate, vinca drug or vehicle control (sterile water). Dosages of the conjugate and vinca drug are initially the maximum non-lethal amount, but may be subsequently titrated lower. Identical doses are administered at 24 hour intervals for 5 days. After 10 days, blood samples are removed from the mice and the serum level of PSA is determined. Similar serum PSA levels are determined at 5-10 day intervals. At the end of 5.5 weeks the mice are sacrificed and weights of any tumors present are measured and serum PSA again determined. The animals' weights are determined at the beginning and end of the assay.

Protocol B:

Ten days after cell inoculation, blood samples are removed from the animals and serum levels of PSA are determined. Animals are then grouped according to their PSA serum levels. At 14-15 days after cell inoculation, the animals are dosed with a 0.1-0.5 mL volume of test conjugate, vinca drug or vehicle control (sterile water). Dosages of the conjugate and vinca drug are initially the maximum non-lethal amount, but may be subsequently titrated lower. Identical doses are administered at 24 hour intervals for 5 days. Serum PSA levels are determined at 5-10 day intervals. At the end of 5.5 weeks the mice are sacrificed, weights of any tumors present are measured and serum PSA again determined. The animals' weights are determined at the beginning and end of the assay.

### Example 6

In vitro determination of proteolytic cleavage of conjugates by endogenous non-PSA proteases

Step A: Preparation of proteolytic tissue extracts

All procedures are carried out at 4 C. Appropriate animals are sacrificed and the relevant tissues are isolated and stored in liquid nitrogen. The frozen tissue is pulverized using a mortar and pestle and the pulverized tissue is transfered to a Potter-Elvejeh homogenizer and 2 volumes of Buffer A (50 mM Tris containing 1.15% KCl, pH 7.5) are added. The tissue is then disrupted with 20 strokes using first a lose fitting and then a tight fitting pestle. The homogenate is centrifuged at 10,000×g in a swinging bucket rotor (HB4-5), the pellet is discarded and the re-supernatant centrifuged at 100,000×g (Ti 70). The supernatant (cytosol) is saved.

The pellet is resuspended in Buffer B (10 mM EDTA containing 1.15% KCl, pH 7.5) using the same volume used in step as used above with Buffer A. The suspension is homogenized in a dounce homogenizer and the solution centrifuged at 100,000×g. The supernatant is discarded and the pellet resuspended in Buffer C (10 mM potassium phosphate buffer containing 0.25 M sucrose, pH 7.4), using 1/2 the volume used above, and homogenized with a dounce homogenizer.

Protein content of the two solutions (cytosol and membrane) is determine using the Bradford assay. Assay aliquots are then removed and frozen in liquid N₂. The aliquots are stored at −70° C.

Step B: Proteolytic Cleavage Assay

For each time point, 20 microgram of peptide-vinca drug conjugate and 150 micrograms of tissue protein, prepared as described in Step A and as determined by Bradford in reaction buffer are placed in solution of final volume of 200 microliters in buffer (50 mM TRIS, 140 mM NaCl, pH 7.2). Assay reactions are run for 0, 30, 60, 120, and 180 minutes and are then quenched with 9 microliters of 0.1 M ZnCl₂ and immediately placed in boiling water for 90 seconds. Reaction products are analyzed by HPLC using a VYDAC C18 15 cm column in water/acetonitrile (5% to 50% acetonitrile over 30 minutes).

What is claimed is:

1. A conjugate which is useful for the treatment of prostate cancer which comprises a cytotoxic agent attached to an oligopeptide, wherein the oligopeptide comprises a sequence of amino acids that is selectively proteolytically cleaved by free prostate specific antigen and wherein the means of attachment is through a hydroxyalkyl-amino chemical linker which is optionally substituted, or the pharmaceutically acceptable salt thereof.

2. The conjugate according to claim 1 wherein the oligopeptide is attached to the chemical linker by an ester bond with that bond comprising the hydroxyl moiety of the chemical linker.

3. The conjugate according to claim 1 wherein the cytotoxic agent is a vinca alkaloid cytotoxic agent.

4. The conjugate according to claim 3 wherein the cytotoxic agent is selected from vinblastine and 4-desacetylvinblastine.

5. A conjugate of the formula I: 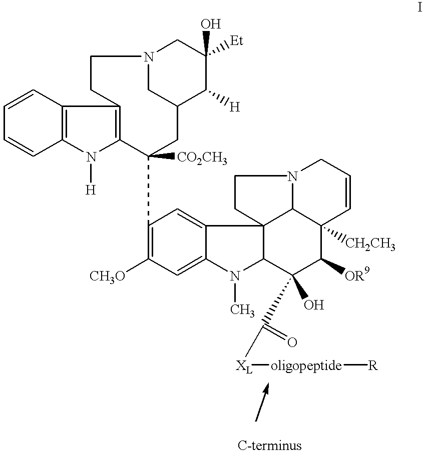
wherein:
oligopeptide is an oligopeptide which is specifically recognized by the free prostate specific antigen (PSA) and is capable of being proteolytically cleaved by the enzymatic activity of the free prostate specific antigen,
X_{L} is selected from —NH—(CR³₂)ᵤ(CR⁴₂)ᵥ—O— and 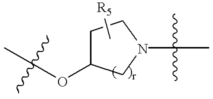
R is selected from
a) hydrogen,
b) —(C═O)R^{1a},
c) 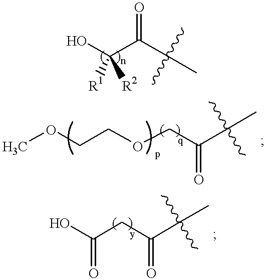
f) ethoxysquarate; and
g) cotininyl;
R¹ and R² are independently selected from:
a) hydrogen,
b) unsubstituted or substituted aryl, unsubstituted or substituted heterocycle, C₃-C₁₀cycloalkyl, C₂-C₆alkenyl, C₂-C₆alkynyl, halogen, C₁-C₆perfluoroalkyl, R⁶O—, R⁶C(O)NR⁶—, (R⁶)₂NC(O)—, R⁶₂N—C(NR⁶)—, R⁷S(O)₂NH, CN, NO₂, R⁶C(O)—, N₃, —N(R⁶)₂, or R⁷OC(O)NR⁶—,
c) unsubstituted C₁-C₆alkyl,
d) substituted C₁-C₆alkyl wherein the substituent on the substituted C₁-C₆alkyl is selected from unsubstituted or substituted aryl, unsubstituted or substituted heterocyclic, C₃-C₁₀cycloalkyl, C₂-C₆alkenyl, C₂-C₆alkynyl, R⁶O—, R⁷S(O)₂NH, R⁶C(O)NR⁶—, (R⁶)₂NC(O)—, R⁶₂N—C(NR⁶)—, CN, R⁶C(O)—, N₃, —N(R⁶)₂, and R⁷OC(O)—NR⁶—; or
R¹ and R² are combined to form —(CH₂)ₛ— wherein one of the carbon atoms is optionally replaced by a moiety selected from: O, S(O)ₘ, —NC(O)—, NH and —N(COR⁷)—;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl,
R³ and R⁴ are independently selected from: hydrogen, C₁-C₆-alkyl, hydroxylated C₃-C₈-cycloalkyl, polyhydroxylated C₃-C₈-cycloalkyl, hydroxylated aryl, polyhydroxylated aryl and aryl, or
one R³ and one R⁴ are combined to form a —(CH₂)_{w}—, which is unsubstituted or substituted with one or two substituents selected from OH and C₁-C₆alkyl; or
an R³ is combined with another R³ on the same carbon to form a —(CH₂)ₓ—; or
an R⁴ is combined with another R⁴ on the same carbon to form a —(CH₂)ₓ—;
R⁵ is selected from OH and C₁-C₆alkyl;
R⁶ is selected from: hydrogen, aryl, substituted aryl, heterocycle, substituted heterocycle, C₁-C₆alkyl and C₃-C₁₀cycloalkyl;
R⁷ is selected from: aryl, substituted aryl, heterocycle, substituted heterocycle, C₁-C₆alkyl and C₃-C₁₀cycloalkyl;
R⁹ is hydrogen, (C₁-C₃alkyl)—CO, or chlorosubstituted (C₁-C₃alkyl)—CO;
n is 1, 2, 3 or 4;
p is zero or an integer between 1 and 100;
q is 0 or 1, provided that if p is zero, q is 1;
r is 1, 2 or 3;
s is 4, 5 or 6;
t is 3 or 4;
u and v are independently selected from: 0, 1, 2 or 3;
w is 2, 3 or 4;
x is 3, 4 or 5;
y is 1, 2 or 3;
or a pharmaceutically acceptable salt thereof.

6. The conjugate according to claim 5 wherein:
oligopeptide is an oligomer that comprises an amino acid sequence selected from:
a) AsnLysIleSerTyrGln/Ser (SEQ.ID.NO.: 1),
b) LysIleSerTyrGln/Ser (SEQ.ID.NO.: 2),
c) AsnLysIleSerTyrTyr/Ser (SEQ.ID.NO.: 3),
d) AsnLysAlaSerTyrGln/Ser (SEQ.ID.NO.: 4),
e) SerTyrGln/SerSer (SEQ.ID.NO.: 5);
f) LysTyrGln/SerSer (SEQ.ID.NO.: 6);
g) hArgTyrGln/SerSer (SEQ.ID.NO.: 7);
h) hArgChaGln/SerSer (SEQ.ID.NO.: 8);
i) TyrGln/SerSer (SEQ.ID.NO.: 9);
j) TyrGln/SerLeu (SEQ.ID.NO.: 10);
k) TyrGln/SerNle (SEQ.ID.NO.: 11);
l) ChgGln/SerLeu (SEQ.ID.NO.: 12);
m) ChgGln/SerNle (SEQ.ID.NO.: 13);
n) SerTyrGln/Ser (SEQ.ID.NO.: 14);
o) SerChgGln/Ser (SEQ.ID.NO.: 15);
p) SerTyrGln/SerVal (SEQ.ID.NO.: 16);
q) SerChgGln/SerVal (SEQ.ID.NO.: 17);
r) SerTyrGln/SerLeu (SEQ.ID.NO.: 18);
s) SerChgGln/SerLeu (SEQ.ID.NO.: 19);
t) HaaXaaSerTyrGln/Ser (SEQ.ID.NO.: 20);
u) HaaXaaLysTyrGln/Ser (SEQ.ID.NO.: 21);
v) HaaXaahArgTyrGln/Ser (SEQ.ID.NO.: 22);
w) HaaXaahArgChaGln/Ser (SEQ.ID.NO.: 23);
x) HaaTyrGln/Ser (SEQ.ID.NO.: 24);
y) HaaXaaSerChgGln/Ser (SEQ.ID.NO.: 25);
z) HaaChgGln/Ser (SEQ.ID.NO.: 26);
aa) SerChgGln/SerSer (SEQ.ID.NO.: 106);
bb) SerChgGln/SerPro (SEQ.ID.NO.: 107); and
cc) SerChgGln/SerAbu (SEQ.ID.NO.: 108);
wherein Haa is a cyclic amino acid substituted with a hydrophilic moiety, hArg is homoarginine, Xaa is any amino acid, Cha is cyclohexylalanine, Abu is 2-aminobutyric acid and Chg is cyclohexylglycine;
or an optical isomer thereof.

7. The conjugate according to claim 6 wherein:
Xaa is alanine, serine or isoleucine; and
Haa is trans-4-hydroxy-L-proline;
or an optical isomer thereof.

8. The conjugate according to claim 5 wherein:
X_{L} is selected from the following group: 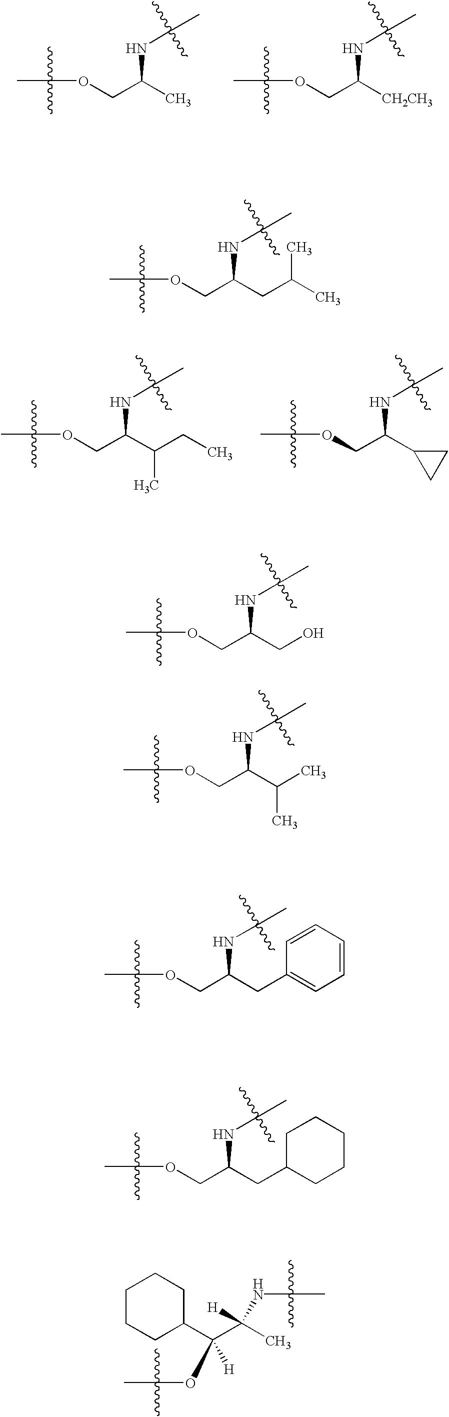
or an optical isomer thereof.

9. The conjugate according to claim 5 which is selected from:

| | SEQ. |
|---|---|
| PEPTIDE-VIN CONJUGATE | ID. NO. |
| Ac-(4-trans-L-Hyp)SSChgQ-SPheol-(dAc)-VIN | 90 |
| Ac-4-trans-L-HypSSChgQS-cyclopropylalaninol- | 91 |
| (dAc)-VIN | |
| Ac-4-trans-L-HypSSChgQS-cyclohexylalaninol- | 92 |
| (dAc)-VIN | |
| Ac-4-trans-L-HypSSChgQS-valinol-(dAc)-VIN | 93 |
| Ac-4-trans-L-HypSSChgQS-(HCAP)-(dAc)-VIN | 82 |
| TFA salt | |
| Ac-4-trans-L-HypSSChgQS-O-3(R)pyrrolidine- | 82 |
| (HCAP)-(dAc)-VIN | |
| Ac-4-trans-L-HypSSChgQ-SS-(HCAP)-(dAc)- | 83 |
| VIN | |
| N-hydroxyacetyl-AbuSSChgQ-SP-(HCAP)- | 85 |
| (dAc)-VIN | |
| Ac-SSChgQ-SP-(HCAP)-(dAc)-VIN | 86 |
| Ac-AbuSSChgQ-SP-(HCAP)-(dAc)-VIN | 84 |
| Ac-SChgQ-SP-(HCAP)-(dAc)-VIN | 94 |
| Ac-AbuSChgQ-SP-(HCAP)-(dAc)-VIN | 95 |
| Ac-SChgQSS-Sar-(HCAP)-dAc-VIN | 96 |
| Ac-SChgQS-Abu-(HCAP)-VIN | 97 |
| Ac-SChgQ-SS(4-trans-L-Hyp)-(HCAP)-dAc-VIN | 98 |
| Ac-SChgQSS(PIP)-(HCAP)-dAc-VIN | 99 |
| Ac-SChgQSS(HCAP)-dAc-VIN | 100 |
| Ac-SChgQSS-gammaAbu-(HCAP)-dAc-VIN | 101 |
| Ac-4-trans-L-HypSSChgQSP(HCAP)-VIN | 102 |
| Ac-SSChgQ-SSP-(HCAP)-dAc-VIN | 103 |
| Ac-SChgQ-SSP-(HCAP)-VIN | 104 |
| Ac-AbuSSChgQ-S-(HCAP)-VIN | 105 |

10. A compound which is selected from: 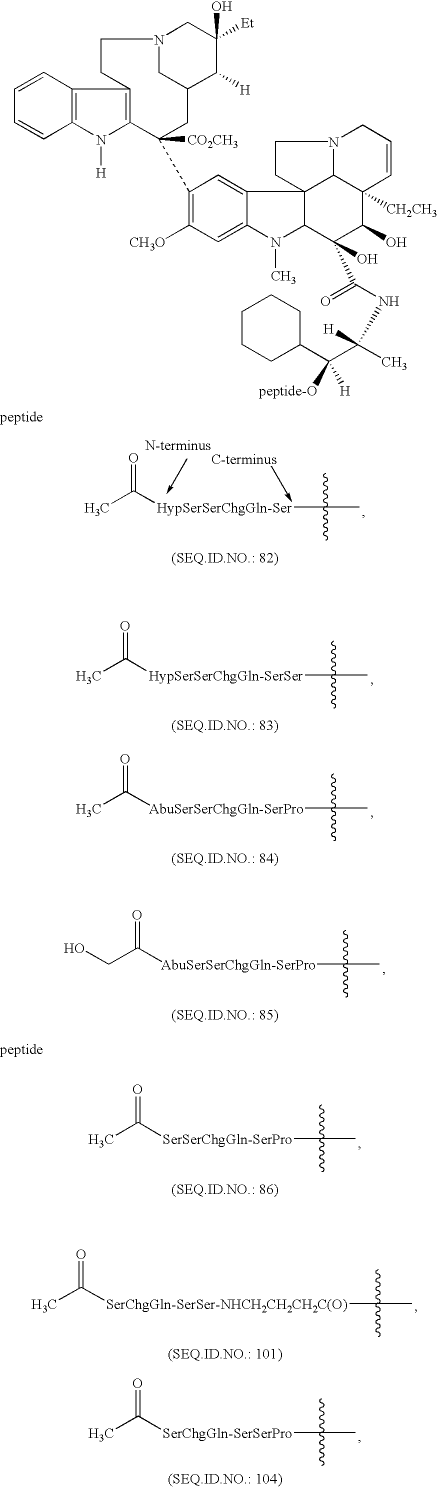
or the pharmaceutically acceptable salt or optical isomer thereof.

11. A pharmaceutical composition comprising a pharmaceutical carrier, and dispersed therein, a therapeutically effective amount of a compound of claim 1.

12. A pharmaceutical composition comprising a pharmaceutical carrier, and dispersed therein, a therapeutically effective amount of a compound of claim 5.

13. A pharmaceutical composition comprising a pharmaceutical carrier, and dispersed therein, a therapeutically effective amount of a compound of claim 10.

14. A method for treating prostate cancer which comprises administering to a mammal in need thereof a therapeutically effective amount of a composition of claim 11.

15. A method for treating prostate cancer which comprises administering to a mammal in need thereof a therapeutically effective amount of a composition of claim 12.

16. A method for treating prostate cancer which comprises administering to a mammal in need thereof a therapeutically effective amount of a composition of claim 13.

17. A method for treating benign prostatic hyperplasia which comprises administering to a mammal in need thereof a therapeutically effective amount of a composition of claim 11.

18. A pharmaceutical composition made by combining the compound of claim 1 and a pharmaceutically acceptable carrier.

19. A process for making a pharmaceutical composition comprising combining a compound of claim 1 and a pharmaceutically acceptable carrier.

20. A compound of the formula II: 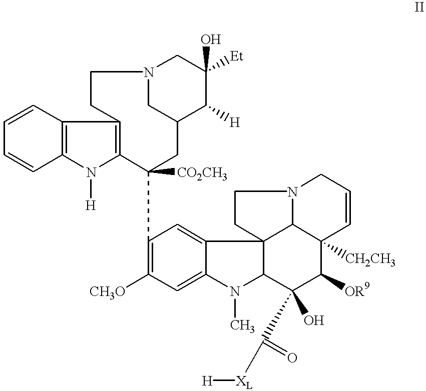
wherein:
X_{L} is selected from —NH—(CR³₂)ᵤ(CR⁴₂)ᵥ—O— and 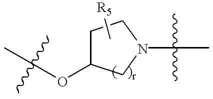
R³ and R⁴ are independently selected from: hydrogen, C₁-C₆-alkyl, hydroxylated C₃-C₈-cycloalkyl, polyhydroxylated C₃-C₈-cycloalkyl, hydroxylated aryl, polyhydroxylated aryl and aryl, or
one R³ and one R⁴ are combined to form a —(CH₂)_{w}—, which is unsubstituted or substituted with one or two substituents selected from OH and C₁-C₆alkyl; or
an R³ is combined with another R³ on the same carbon to form a —(CH₂)ₓ—; or
an R⁴ is combined with another R⁴ on the same carbon to form a —(CH₂)ₓ—;
R⁵ is selected from OH and C₁-C₆alkyl;
R⁹ is hydrogen, (C₁-C₃alkyl)—CO, or chlorosubstituted (C₁-C₃alkyl)—CO; and
r is 1, 2 or 3;
u and v are independently selected from: 0, 1, 2 or 3;
w is 2, 3 or 4;
x is 3, 4 or 5;
or the pharmaceutically acceptable salt or optical isomer thereof.

21. The compound according to claim 20 selected from: 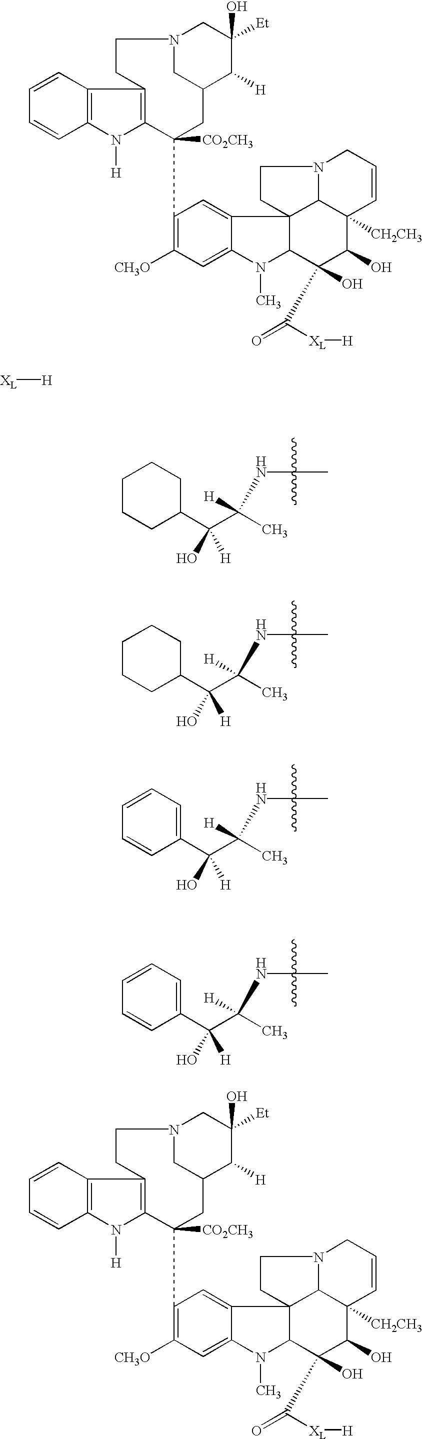
or the pharmaceutically acceptable salt thereof.

22. A pharmaceutical composition comprising a pharmaceutical carrier, and dispersed therein, a therapeutically effective amount of a compound of claim 20.

23. A pharmaceutical composition comprising a pharmaceutical carrier, and dispersed therein, a therapeutically effective amount of a compound of claim 21.

24. A method for treating cancer which comprises administering to a mammal in need thereof a therapeutically effective amount of a composition of claim 22.

25. A method for treating cancer which comprises administering to a mammal in need thereof a therapeutically effective amount of a composition of claim 23.


SEQUENCE LISTING (108 sequences)

SEQ ID NO: 1 - protein, 7 aa, Artificial Sequence
  completely synthetic amino acid sequence
NKISYQS

SEQ ID NO: 2 - protein, 6 aa, Artificial Sequence
  completely synthetic amino acid sequence
KISYQS

SEQ ID NO: 3 - protein, 7 aa, Artificial Sequence
  completely synthetic amino acid sequence
NKISYYS

SEQ ID NO: 4 - protein, 7 aa, Artificial Sequence
  completely synthetic amino acid sequence
NKASYQS

SEQ ID NO: 5 - protein, 5 aa, Artificial Sequence
  completely synthetic amino acid sequence
SYQSS

SEQ ID NO: 6 - protein, 5 aa, Artificial Sequence
  completely synthetic amino acid sequence
KYQSS

SEQ ID NO: 7 - protein, 5 aa, Artificial Sequence
  completely synthetic amino acid sequence
XYQSS

SEQ ID NO: 8 - protein, 5 aa, Artificial Sequence
  completely synthetic amino acid sequence
XXQSS

SEQ ID NO: 9 - protein, 4 aa, Artificial Sequence
  completely synthetic amino acid sequence
YQSS

SEQ ID NO: 10 - protein, 4 aa, Artificial Sequence
  completely synthetic amino acid sequence
YQSL

SEQ ID NO: 11 - protein, 4 aa, Artificial Sequence
  completely synthetic amino acid sequence
YQSL

SEQ ID NO: 12 - protein, 4 aa, Artificial Sequence
  completely synthetic amino acid sequence
XQSL

SEQ ID NO: 13 - protein, 4 aa, Artificial Sequence
  completely synthetic amino acid sequence
XQSL

SEQ ID NO: 14 - protein, 4 aa, Artificial Sequence
  completely synthetic amino acid sequence
SYQS

SEQ ID NO: 15 - protein, 4 aa, Artificial Sequence
  completely synthetic amino acid sequence
SXQS

SEQ ID NO: 16 - protein, 5 aa, Artificial Sequence
  completely synthetic amino acid sequence
SYQSV

SEQ ID NO: 17 - protein, 5 aa, Artificial Sequence
  completely synthetic amino acid sequence
SXQSV

SEQ ID NO: 18 - protein, 5 aa, Artificial Sequence
  completely synthetic amino acid sequence
SYQSL

SEQ ID NO: 19 - protein, 5 aa, Artificial Sequence
  completely synthetic amino acid sequence
SXQSL

SEQ ID NO: 20 - protein, 6 aa, Artificial Sequence
  completely synthetic amino acid sequence
XXSYQS

SEQ ID NO: 21 - protein, 6 aa, Artificial Sequence
  completely synthetic amino acid sequence
XXKYQS

SEQ ID NO: 22 - protein, 6 aa, Artificial Sequence
  completely synthetic amino acid sequence
XXXYQS

SEQ ID NO: 23 - protein, 6 aa, Artificial Sequence
  completely synthetic amino acid sequence
XXXXQS

SEQ ID NO: 24 - protein, 4 aa, Artificial Sequence
  completely synthetic amino acid sequence
XYQS

SEQ ID NO: 25 - protein, 6 aa, Artificial Sequence
  completely synthetic amino acid sequence
XXSXQS

SEQ ID NO: 26 - protein, 4 aa, Artificial Sequence
  completely synthetic amino acid sequence
XXQS

SEQ ID NO: 27 - protein, 6 aa, Artificial Sequence
  completely synthetic amino acid sequence
SSYQSV

SEQ ID NO: 28 - protein, 6 aa, Artificial Sequence
  completely synthetic amino acid sequence
SSXQSV

SEQ ID NO: 29 - protein, 6 aa, Artificial Sequence
  completely synthetic amino acid sequence
SSYQSL

SEQ ID NO: 30 - protein, 6 aa, Artificial Sequence
  completely synthetic amino acid sequence
SSXQSL

SEQ ID NO: 31 - protein, 6 aa, Artificial Sequence
  completely synthetic amino acid sequence
SSYQSS

SEQ ID NO: 32 - protein, 6 aa, Artificial Sequence
  completely synthetic amino acid sequence
SSXQSS

SEQ ID NO: 33 - protein, 6 aa, Artificial Sequence
  completely synthetic amino acid sequence
SSYQSP

SEQ ID NO: 34 - protein, 6 aa, Artificial Sequence
  completely synthetic amino acid sequence
SSXQSP

SEQ ID NO: 35 - protein, 6 aa, Artificial Sequence
  completely synthetic amino acid sequence
XSSYQS

SEQ ID NO: 36 - protein, 6 aa, Artificial Sequence
  completely synthetic amino acid sequence
XSSXQS

SEQ ID NO: 37 - protein, 6 aa, Artificial Sequence
  completely synthetic amino acid sequence
ASYQSV

SEQ ID NO: 38 - protein, 6 aa, Artificial Sequence
  completely synthetic amino acid sequence
ASXQSV

SEQ ID NO: 39 - protein, 6 aa, Artificial Sequence
  completely synthetic amino acid sequence
ASYQSL

SEQ ID NO: 40 - protein, 6 aa, Artificial Sequence
  completely synthetic amino acid sequence
ASXQSL

SEQ ID NO: 41 - protein, 6 aa, Artificial Sequence
  completely synthetic amino acid sequence
XASYQS

SEQ ID NO: 42 - protein, 6 aa, Artificial Sequence
  completely synthetic amino acid sequence
XASXQS

SEQ ID NO: 43 - protein, 6 aa, Artificial Sequence
  completely synthetic amino acid sequence
SSXQSL

SEQ ID NO: 44 - protein, 6 aa, Artificial Sequence
  completely synthetic amino acid sequence
SSXQSV

SEQ ID NO: 45 - protein, 6 aa, Artificial Sequence
  completely synthetic amino acid sequence
SSXQSP

SEQ ID NO: 46 - protein, 6 aa, Artificial Sequence
  completely synthetic amino acid sequence
SSXQSS

SEQ ID NO: 47 - protein, 7 aa, Artificial Sequence
  completely synthetic amino acid sequence
SSSXQSL

SEQ ID NO: 48 - protein, 7 aa, Artificial Sequence
  completely synthetic amino acid sequence
SSSXQSV

SEQ ID NO: 49 - protein, 7 aa, Artificial Sequence
  completely synthetic amino acid sequence
SSSXQSP

SEQ ID NO: 50 - protein, 7 aa, Artificial Sequence
  completely synthetic amino acid sequence
SSSXQSS

SEQ ID NO: 51 - protein, 7 aa, Artificial Sequence
  completely synthetic amino acid sequence
SASXQSL

SEQ ID NO: 52 - protein, 7 aa, Artificial Sequence
  completely synthetic amino acid sequence
SASXQSV

SEQ ID NO: 53 - protein, 7 aa, Artificial Sequence
  completely synthetic amino acid sequence
XSSXQSL

SEQ ID NO: 54 - protein, 7 aa, Artificial Sequence
  completely synthetic amino acid sequence
XSSXQSV

SEQ ID NO: 55 - protein, 7 aa, Artificial Sequence
  completely synthetic amino acid sequence
XSSYQSV

SEQ ID NO: 56 - protein, 7 aa, Artificial Sequence
  completely synthetic amino acid sequence
XSSYQSL

SEQ ID NO: 57 - protein, 7 aa, Artificial Sequence
  completely synthetic amino acid sequence
XSSXQSV

SEQ ID NO: 58 - protein, 7 aa, Artificial Sequence
  completely synthetic amino acid sequence
XSSXQSL

SEQ ID NO: 59 - protein, 7 aa, Artificial Sequence
  completely synthetic amino acid sequence
XSSXQSS

SEQ ID NO: 60 - protein, 7 aa, Artificial Sequence
  completely synthetic amino acid sequence
XSSXQSS

SEQ ID NO: 61 - protein, 7 aa, Artificial Sequence
  completely synthetic amino acid sequence
XSSXQSP

SEQ ID NO: 62 - protein, 7 aa, Artificial Sequence
  completely synthetic amino acid sequence
XSSXQSP

SEQ ID NO: 63 - protein, 7 aa, Artificial Sequence
  completely synthetic amino acid sequence
XASXQSV

SEQ ID NO: 64 - protein, 7 aa, Artificial Sequence
  completely synthetic amino acid sequence
XASXQSL

SEQ ID NO: 65 - protein, 7 aa, Artificial Sequence
  completely synthetic amino acid sequence
XSSYQSV

SEQ ID NO: 66 - protein, 7 aa, Artificial Sequence
  completely synthetic amino acid sequence
XSSYQSL

SEQ ID NO: 67 - protein, 7 aa, Artificial Sequence
  completely synthetic amino acid sequence
XSAXQSL

SEQ ID NO: 68 - protein, 6 aa, Artificial Sequence
  completely synthetic amino acid sequence
SXXQSL

SEQ ID NO: 69 - protein, 5 aa, Artificial Sequence
  completely synthetic amino acid sequence
XXQSL

SEQ ID NO: 70 - protein, 7 aa, Artificial Sequence
  completely synthetic amino acid sequence
NRISYQS

SEQ ID NO: 71 - protein, 7 aa, Artificial Sequence
  completely synthetic amino acid sequence
NKVSYQS

SEQ ID NO: 72 - protein, 8 aa, Artificial Sequence
  completely synthetic amino acid sequence
NKMSYQSS

SEQ ID NO: 73 - protein, 8 aa, Artificial Sequence
  completely synthetic amino acid sequence
NKLSYQSS

SEQ ID NO: 74 - protein, 7 aa, Artificial Sequence
  completely synthetic amino acid sequence
NKISYQS

SEQ ID NO: 75 - protein, 8 aa, Artificial Sequence
  completely synthetic amino acid sequence
QKISYQSS

SEQ ID NO: 76 - protein, 7 aa, Artificial Sequence
  completely synthetic amino acid sequence
NXISYQS

SEQ ID NO: 77 - protein, 7 aa, Artificial Sequence
  completely synthetic amino acid sequence
NXVSYQS

SEQ ID NO: 78 - protein, 7 aa, Artificial Sequence
  completely synthetic amino acid sequence
XASYQSS

SEQ ID NO: 79 - protein, 7 aa, Artificial Sequence
  completely synthetic amino acid sequence
XASYQSS

SEQ ID NO: 80 - protein, 5 aa, Artificial Sequence
  completely synthetic amino acid sequence
XSXQS

SEQ ID NO: 81 - protein, 7 aa, Artificial Sequence
  completely synthetic amino acid sequence
XASXQSS

SEQ ID NO: 82 - protein, 6 aa, Artificial Sequence
  completely synthetic amino acid sequence
XASXQS

SEQ ID NO: 83 - protein, 7 aa, Artificial Sequence
  completely synthetic amino acid sequence
XSSXQSS

SEQ ID NO: 84 - protein, 7 aa, Artificial Sequence
  completely synthetic amino acid sequence
XSSXQSP

SEQ ID NO: 85 - protein, 7 aa, Artificial Sequence
  completely synthetic amino acid sequence
XSSXQSP

SEQ ID NO: 86 - protein, 6 aa, Artificial Sequence
  completely synthetic amino acid sequence
XSXQSP

SEQ ID NO: 87 - protein, 5 aa, Artificial Sequence
  completely synthetic amino acid sequence
SSSXQ

SEQ ID NO: 88 - protein, 6 aa, Artificial Sequence
  completely synthetic amino acid sequence
XSSXQS

SEQ ID NO: 89 - protein, 7 aa, Artificial Sequence
  completely synthetic amino acid sequence
XSSXQSX

SEQ ID NO: 90 - protein, 6 aa, Artificial Sequence
  completely synthetic amino acid sequence
XSSXQS

SEQ ID NO: 91 - protein, 6 aa, Artificial Sequence
  completely synthetic amino acid sequence
XSSXQX

SEQ ID NO: 92 - protein, 7 aa, Artificial Sequence
  completely synthetic amino acid sequence
XSSXQSX

SEQ ID NO: 93 - protein, 6 aa, Artificial Sequence
  completely synthetic amino acid sequence
XSSXQX

SEQ ID NO: 94 - protein, 5 aa, Artificial Sequence
  completely synthetic amino acid sequence
XXQSX

SEQ ID NO: 95 - protein, 4 aa, Artificial Sequence
  completely synthetic amino acid sequence
XQSX

SEQ ID NO: 96 - protein, 6 aa, Artificial Sequence
  completely synthetic amino acid sequence
XXQSSX

SEQ ID NO: 97 - protein, 5 aa, Artificial Sequence
  completely synthetic amino acid sequence
XXQSX

SEQ ID NO: 98 - protein, 6 aa, Artificial Sequence
  completely synthetic amino acid sequence
XXQSSX

SEQ ID NO: 99 - protein, 6 aa, Artificial Sequence
  completely synthetic amino acid sequence
XXQSSX

SEQ ID NO: 100 - protein, 5 aa, Artificial Sequence
  completely synthetic amino acid sequence
XXQSX

SEQ ID NO: 101 - protein, 6 aa, Artificial Sequence
  completely synthetic amino acid sequence
XXQSSX

SEQ ID NO: 102 - protein, 7 aa, Artificial Sequence
  completely synthetic amino acid sequence
XSSXQSX

SEQ ID NO: 103 - protein, 7 aa, Artificial Sequence
  completely synthetic amino acid sequence
XSXQSSX

SEQ ID NO: 104 - protein, 6 aa, Artificial Sequence
  completely synthetic amino acid sequence
SXQSSX

SEQ ID NO: 105 - protein, 6 aa, Artificial Sequence
  completely synthetic amino acid sequence
XSSXQX

SEQ ID NO: 106 - protein, 5 aa, Artificial Sequence
  completely synthetic amino acid sequence
SXQSS

SEQ ID NO: 107 - protein, 5 aa, Artificial Sequence
  completely synthetic amino acid sequence
SXQSP

SEQ ID NO: 108 - protein, 5 aa, Artificial Sequence
  completely synthetic amino acid sequence
SXQSX